(19) United States
(12) Patent Application Publication
(10) Pub. No.: US 2025/0049598 A1
(43) Pub. Date: Feb. 13, 2025

(54) SYSTEMS AND METHODS FOR TREATING AN AORTIC TEAR OR DISSECTION

(71) Applicant: Nectero Medical, Inc., Tempe, AZ (US)

(72) Inventors: Kelvin Ning, Scottsdale, AZ (US); Adam Charles Gold, Tempe, AZ (US); Pankti Nimishkumar Shah, Tempe, AZ (US); Natalie Marie Stevens, Tempe, AZ (US); Robert Whirley, Santa Rosa, CA (US); Venkatesh Ramaiah, Scottsdale, AZ (US)

(21) Appl. No.: 18/667,538

(22) Filed: May 17, 2024

Related U.S. Application Data
(63) Continuation of application No. PCT/US2022/080344, filed on Nov. 22, 2022.
(60) Provisional application No. 63/283,168, filed on Nov. 24, 2021.

(51) Int. Cl. A61F 2/95; A61F 2/82; A61M 5/00; A61M 25/10

(52) U.S. Cl. CPC A61F 2/95; A61M 5/007; A61M 25/10; A61F 2002/821; A61F 2002/823; A61M 2025/105; A61M 2210/127

(57) ABSTRACT

Systems and methods for treatment of an aortic tear or dissection (such as in the thoracic aortic region) are provided. Some methods disclosed herein include inserting an endoprosthesis near an inlet flap of an aortic tear or dissection, inserting an infusion catheter near the inlet flap of the aortic tear or dissection, positioning said infusion catheter between an outer surface of the endoprosthesis and an aortic wall, expanding the endoprosthesis so that the infusion catheter is in contact with aortic tissue near the inlet flap, infusing a stabilization treatment compound through the infusion catheter, wherein the stabilization treatment compound comprises a contrast media agent, a stabilizing agent, and one or more excipients, and removing the infusion catheter.

### INCORPORATION BY REFERENCE TO ANY PRIORITY APPLICATIONS

This application claims priority to Int. Pat. App. No. PCT/US2022/080344, filed Nov. 22, 2022, which claims priority to U.S. Provisional Application No. 63/283,168 filed Nov. 24, 2021, the entire contents of which are hereby incorporated by reference herein. Any and all applications for which a foreign or domestic priority claim is identified in the Application Data Sheet as filed with the present application are hereby incorporated by reference under 37 CFR 1.57.

### FIELD

The present disclosure generally relates to systems and methods for treatment of an aortic tear or dissection. The systems and methods for treatment of an aortic tear or dissection may include delivery of a stabilizing compound.

### BACKGROUND

Diseases of the aorta have several different clinical manifestations. In the abdominal aorta, aortic diseases manifest predominantly in the form of an aneurysm, or Abdominal Aortic Aneurysm (AAA). In the thoracic aorta, stresses more commonly result in a tear in the wall of the aorta, which in turn can propagate as a dissection and potentially narrow the true lumen along a thoracic portion of the aorta. Alternatively, just like an AAA, the thoracic aorta may also form an aneurysm, or Thoracic Aortic Aneurysm (TAA). Dissections may also form in or propagate into the ascending thoracic aorta and the aortic arch. The clinical risk of these diseases is rupture of the aorta; the compromised aortic tissue may not be able to withstand the shear stress imposed by the hemodynamic forces present.

Acute or subacute thoracic aortic dissections are typically characterized by an inlet tear. This inlet tear allows the hemodynamic pressure to dissect, or separate, the vascular wall, thereby creating a false lumen. Usually, there is potentially a point of re-entry of this tear into the true lumen, thereby making the false lumen an alternative flow channel to the true lumen. In current treatments, typically a 10 to 15 cm stent (covered or uncovered) is placed in the true lumen over the inlet flap and along a distally extending portion of the dissection. Pressure in the false lumen is reduced, but retrograde flow may continue to perfuse the false lumen. Known complications include extension of the dissection proximally and/or distally from the end of the implanted prosthesis, arising both from the mechanical stiffness discontinuities at the ends of the implanted prosthesis and from the traumatic deployment effects on the diseased aortic wall.

Thoracic Aortic Aneurysms (TAAs) can form on the ascending or descending portions of the aorta, or both. Aneurysms less than 5 cm in diameter have significantly less risk of rupture. Aneurysms in the ascending thoracic aorta and the aortic arch pose an additional risk because these regions of the aorta feed into the arteries that lead to the upper limbs, including the brain. Perfusion needs to be maintained to keep the brain oxygenated.

To date, the general approach for treatment has been replacement of the diseased segment of aorta with a surgical graft, or implantation of an endoprosthesis to reduce the pressure experienced by the compromised aortic tissue. These implants bridge healthy portions of the aorta with a structure that aims to relieve the local hemodynamically induced stresses in the diseased portions of the aortic wall. When used for aortic dissections, these implants function secondarily to provide mechanical support to the true lumen and mechanical compression to the false lumen.

Acutely, these endoprosthetic implants address the local pressure and risk of wall rupture, but do not address the disease itself. The diseased portions of the aorta may continue to degenerate and extend the distance from healthy tissue to healthy-tissue segments that the original prosthetic implant bridged. As a result, continued monitoring is required for these patients even after implantation of the prosthesis.

### SUMMARY OF THE DISCLOSURE

In a first aspect, a method of treating an aortic tear or dissection (e.g., in a thoracic aorta portion, in an ascending aorta portion, in a descending aorta portion, in the aortic arch, in the abdominal aorta) is disclosed. The method may include, for example, inserting an endoprosthesis (e.g., an implantable stent) near an inlet flap of an aortic tear or dissection, inserting an infusion catheter near the inlet flap of the aortic tear or dissection, positioning said infusion catheter between an outer surface of the endoprosthesis and an aortic wall, expanding the endoprosthesis so that the infusion catheter is in contact with aortic tissue near the inlet flap, infusing a stabilization treatment compound through the infusion catheter, the stabilization treatment compound including a contrast media agent, a stabilizing agent, and/or one or more excipients, and removing the infusion catheter.

In some implementations, the infusion catheter is placed or introduced prior to the placement (e.g., implantation or introduction) of the endoprosthesis. In some implementations, the endoprosthesis is placed (e.g., implanted or positioned) prior to the placement of the infusion catheter. In some implementations, contact with the aortic wall includes contact with the inlet flap. In some implementations, contact with the aortic wall does not include contact with the inlet flap. In some implementations, the stabilizing agent includes a polyphenolic compound. In some implementations, the infusion catheter includes a perforated, or porous, balloon. In some implementations, the infusion catheter comprises one or more perforated linear catheters (e.g., one, two, three, or more than three catheters).

In another aspect, a method of treating an aortic tear or dissection is disclosed. The method may include, for example, coupling a balloon with a central passage to an endoprosthesis, wherein an outer wall of the balloon includes perforations or pores, inserting said endoprosthesis near an inlet flap of an aortic tear or dissection, expanding the endoprosthesis so that the balloon is in contact with aortic tissue, infusing a stabilization treatment compound through the balloon so that stabilization treatment compound weeps onto aortic tissue, the stabilization treatment compound including a contrast media agent, a stabilizing agent, and/or one or more excipients, and deflating and removing the balloon.

In some implementations, coupling the balloon to the endoprosthesis includes preloading the balloon into a coaxial restraining sleeve and inserting the endoprosthesis through the balloon. In some implementations the method further includes removing the restraining sleeve, thereby allowing the endoprosthesis to push the balloon into position against the aortic wall. In some implementations, the stabilizing agent includes a polyphenolic compound. In some implementations, the one or more excipients include poloxamer.

In another aspect, a method of treating an aortic tear or dissection, wherein the aortic tear or dissection comprises a true lumen of a thoracic aorta, and an aortic wall of the thoracic aorta with an inlet flap leading to a false lumen is disclosed. The method may include, for example, placing a distal end of an infusion catheter inside the false lumen, placing a stent in a true lumen of the thoracic aorta, and delivering a stabilization treatment compound through the infusion catheter into the false lumen. The stabilization treatment compound includes a contrast media agent, a stabilizing agent, and/or one or more excipients.

In some implementations, the one or more excipients include a solid poloxamer configured to biodegrade after at least 15 minutes of residence within the false lumen, wherein degradation of the solid poloxamer facilitates dispersion of the stabilizing agent. In some implementations, the catheter is placed prior to placement of the stent. In some implementations, the stent is placed prior to placement of the infusion catheter. In some implementations, the stabilizing agent includes a polyphenolic compound. In some configurations, the infusion catheter includes a tube with perforations or openings in a distal portion of the infusion catheter. In some configurations, a distal portion of the infusion catheter includes a perforated or porous balloon.

In another aspect, a system for treatment of an aortic tear or dissection is disclosed. The system includes, for example, an endoprosthesis (e.g., a stent), an infusion catheter shaped to facilitate passage through or behind the endoprosthesis and extending beyond a distal end of the endoprosthesis, and a stabilizing treatment compound including a contrast agent, a stabilizing agent, and/or one or more excipients.

In some implementations, the infusion catheter has a spiral curvature formed from shape memory material (e.g., shape memory metal or polymer material) designed to make at least one revolution within a lumen of an aortic aneurysm (e.g., thoracic aortic aneurysm). In some configurations, the infusion catheter includes an internal wire stylet to manipulate and/or provide rigidity to at least a portion of the infusion catheter. In some implementations, the infusion catheter includes a primary tube and a coaxial secondary tube, wherein the secondary tube may slide over the primary tube of the infusion catheter to occlude more proximal perforations or pores. In some implementations, the infusion catheter includes a tube with perforations, openings, or pores in a distal portion of the tube. In some configurations, a distal portion of the infusion catheter includes a perforated balloon. In some instances, the stabilizing treatment compound is in liquid form. In some instances, the stabilizing treatment includes a coating on the endoprosthesis. In some instances, the entire endoprosthesis is coated with the stabilizing treatment compound. In some instances, only a portion of the endoprosthesis is coated with the stabilizing treatment compound.

In another aspect, a method of treating a thoracic aortic aneurysm is disclosed. The method may include, for example, placing a flow diverting catheter within a lumen of a thoracic aorta near a thoracic aortic aneurysm, the flow diverting catheter including an occlusive element on a proximal and/or distal end of the flow diverting catheter, infusing a stabilizing treatment compound to penetrate an aneurysmal wall, the stabilizing treatment compound including a contrast media agent, a stabilizing agent, and one or more excipients, aspirating remaining stabilizing treatment compound, and removing the flow diverting catheter.

In some implementations, the flow diverting catheter comprises an inner scaffold disposed within an outer scaffold disposed within a perforated balloon. In some implementations, the occlusive element includes a balloon. In some implementations, the occlusive element includes a ring covered with a flexible pre-shaped material. In some implementations, the flexible pre-shaped material includes a tapered cone with an open cylindrical outlet. In some implementations, the flow diverting catheter is used to infuse the stabilizing treatment compound. In some implementations, a second catheter is used to infuse the stabilizing treatment compound.

In another aspect, a system for treatment of a thoracic aortic aneurysm is disclosed. The system includes a flow diverting catheter including an occlusive element on a proximal and/or distal end of the flow diverting catheter, and a stabilizing treatment compound including a contrast media agent, a stabilizing agent, and/or one or more excipients.

In some configurations, the occlusive element includes a balloon. In some configurations, the occlusive element is a ring covered with a flexible pre-shaped material. In some configurations, the flexible pre-shaped material includes a tapered cone with an open cylindrical outlet. In some configurations, a second catheter for infusion of the stabilizing treatment compound is also included.

In another aspect, a method of treating an aortic tear or dissection of a subject is provided. In some implementations, the method includes inserting an infusion catheter near an inlet flap of the aortic tear or dissection; expanding the infusion catheter so that the infusion catheter is in contact with aortic tissue near the inlet flap; infusing a stabilization treatment compound through the infusion catheter, the stabilization treatment compound including a contrast media agent, a stabilizing agent, and/or one or more excipients; and removing the infusion catheter from the subject.

In some implementations, the infusion catheter includes two or more elongated perforated balloons. In some implementations, the two or more elongated perforated balloons are coupled to a central tubular lumen.

In some implementations, the infusion catheter includes a rigid material or wire covered with a perforated balloon-like material. In some implementations, the infusion catheter may be actuated to form a spiral, and expanding the infusion catheter includes actuating the infusion catheter to form a spiral. In some implementations, the infusion catheter includes an actuating catheter tube, and a fluid communication tube configured to be actuated to form a spiral around the actuating catheter tube.

In some implementations, the method further includes inserting an endoprosthesis near the inlet flap of an aortic tear or dissection. In some implementations, the stabilizing agent includes 1,2,3,4,6-Pentagalloyl Glucose (PGG) or another PGG compound.

In another aspect, a method of treating an aortic tear or dissection of a subject is provided. In some implementations, the method includes inserting an infusion catheter including two or more elongated balloons near an inlet flap of the aortic tear or dissection, expanding the infusion catheter so that the infusion catheter is in contact with aortic tissue near the inlet flap, wherein the two or more elongated balloons are coated with a stabilization treatment compound including a stabilizing agent and one or more excipients, and removing the infusion catheter from the subject. In some implementations, the infusion catheter includes an inner scaffold or middle structure including a perfusion lumen. In some implementations, the method further includes inserting an endoprosthesis near the inlet flap of an aortic tear or dissection. In some implementations, the stabilizing agent comprises 1,2,3,4,6-Pentagalloyl Glucose (PGG) or another PGG compound.

In another aspect, a method of treating an aortic tear or dissection of a subject is provided herein. In some implementations, the method includes inserting an infusion catheter including a catheter that may be actuated to form a spiral near an inlet flap of the aortic tear or dissection, expanding the infusion catheter by actuating the catheter to form a spiral such that the infusion catheter is in contact with aortic tissue near the inlet flap, infusing a stabilization treatment compound through the infusion catheter, the stabilization treatment compound including a contrast media agent, a stabilizing agent, and one or more excipients, inserting an endoprosthesis within loops of the spiral, removing the infusion catheter from the subject; and deploying the endoprosthesis near the inlet flap of an aortic tear or dissection. In some implementations, the stabilizing agent includes 1,2,3,4,6-Pentagalloyl Glucose (PGG) or another PGG compound.

### BRIEF DESCRIPTION OF THE DRAWINGS

The features and advantages of the systems, devices, and methods described herein will become apparent from the following description, taken in conjunction with the accompanying drawings. These drawings depict only several embodiments in accordance with the disclosure and are not to be considered limiting of its scope. In the drawings, similar reference numbers or symbols typically identify similar components, unless context dictates otherwise. The drawings may not be drawn to scale.

FIG. 1 is a side partial cross-sectional view of an aorta with an aortic tear or dissection (such as in the thoracic aorta region or other aorta region), where an endoprosthesis and an infusion catheter have been placed near the inlet flap of the aortic tear or dissection.

FIG. 2 is a side partial cross-sectional view of an aorta with an aortic tear or dissection, where an infusion catheter comprising a balloon with a central passage coupled to an endoprosthesis has been deployed near the inlet flap of the aortic tear or dissection.

FIG. 3 is a side partial cross-sectional view of an aorta with an aortic tear or dissection, where an endoprosthesis and an infusion catheter have been placed near the inlet flap of the aortic tear or dissection. FIG. 3A is a cross-sectional view of FIG. 3 along an axis perpendicular to a longitudinal axis of the aorta.

FIG. 4 is a side partial cross-sectional view of an aorta with an aortic tear or dissection, where an infusion catheter has been placed in the false lumen through one of the re-entry tears, an endoprosthesis has been placed near the inlet tear, and a second catheter with an internal wire stylet has been placed in the true lumen of the aorta. FIG. 4A is a cross-sectional view of FIG. 4 along an axis perpendicular to a longitudinal axis of the aorta.

FIG. 5 is a side partial cross-sectional view of an aorta with an aortic tear or dissection, where an infusion catheter with an internal wire stylet has been placed within the true lumen of the aorta near the inlet flap and has been manipulated to obtain a spiral shape.

FIG. 6A is a side partial cross-sectional view of an aorta with an aortic tear or dissection, where an infusion catheter including two or more elongated balloons has been placed within the true lumen of the aorta near the inlet flap. FIG. 6B depicts a side view of an infusion catheter including two or more elongated balloons. FIG. 6C is a cross section view of FIG. 6A along an axis perpendicular to a longitudinal axis of the aorta. FIG. 6D is a cross section view along an axis perpendicular to a longitudinal axis of the aorta with an example of an infusion catheter including eight elongated balloons. FIG. 6E is a cross section view along an axis perpendicular to a longitudinal axis of the aorta with a further example of an infusion catheter including eight elongated balloons. FIG. 6F is a side view of a balloon used in an example of an infusion catheter.

FIG. 7A depicts a side partial cross-sectional view of an aorta with an aortic tear or dissection, where an infusion catheter with a first fluid communication tube actuated to form a spiral along the thoracic aortic wall, and second actuating catheter tube. FIG. 7B is a side view of an infusion catheter with a first fluid communication tube actuated to form a spiral along the thoracic aortic wall, and second actuating catheter tube. FIG. 7C is a side view of an infusion catheter of FIG. 7B, where the first fluid communication tube and the second actuating catheter tube are partially enclosed within an introducer sheath.

FIG. 8A depicts a side partial cross-sectional view of an aorta with an aortic tear or dissection, where an infusion catheter delivers a stabilizing compound or agent via a perforated or porous balloon. FIG. 8B depicts an oblique partial cutaway view of the infusion catheter of FIG. 8A. FIG. 8C is a cross section view of FIG. 8A along an axis perpendicular to a longitudinal axis of the aorta.

FIG. 9A depicts a side partial cross-sectional view of an aorta with an aortic tear or dissection, where an infusion catheter delivers a stabilizing compound via a perforated balloon. FIG. 9B depicts an oblique partial cutaway view of the infusion catheter of FIG. 9A.

FIG. 9C is a cross section view of FIG. 9A along an axis perpendicular to a longitudinal axis of the aorta.

FIG. 10A depicts a side partial cross-sectional view of an aorta with an aortic tear or dissection, where a spiral shaped infusion catheter is placed prior to the deployment thoracic endovascular aneurysm repair implant. FIG. 10B depicts a side partial cross-sectional view of an aorta with an aortic tear or dissection, where a spiral shaped infusion catheter is removed.

FIG. 11A depicts a side partial cross-sectional view of an aorta with an aortic tear or dissection, where two or more linear catheters are placed between an implant (e.g., endoprosthesis) and a vessel wall. FIG. 11B depicts a side partial cross-sectional view of an aorta with an aortic tear or dissection, where two or more linear catheters are placed in contact with a vessel wall after an implant (e.g., endoprosthesis) has been deployed.

### DETAILED DESCRIPTION

Throughout the description, including in the Summary and Drawings, reference is made to particular features of the disclosure. It is to be understood that the disclosure in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments, aspects, implementations, configurations, or instances of the inventions, and in the inventions generally.

Unless defined otherwise, all technical and scientific terms used herein have the same meaning as is commonly understood by one of ordinary skill in the art. In the event that there is a plurality of definitions for a term herein, those in this section prevail unless stated otherwise.

Aneurysms of the aorta can be divided into ascending aorta, descending aorta, thoracic, and abdominal. The disease is generally described as a weakening of the vascular wall which is then exacerbated by the high pressures within the aorta. Without intervention, the descending and thoracic aortic aneurysms are prone to dissections and ruptures. An incomplete rupture of the tissue is initiated by a small tear in the intima layer of the vessel wall which allows pressure from the aorta to penetrate and delaminate the media layer of the vessel. This delamination propagates downward until it finds a weak point; this weak point either pushes outward through the adventitia or inward back into the native vessel lumen.

Traditional types of intervention include open surgical or endovascular intervention. For both treatment options, the goal of treatment is to reduce the pressure felt by the native tissue, thereby reducing the risk of rupture. In the case of an open surgical repair, a long incision is made along the aneurysm and opened. Any thrombus or debris is removed prior to sewing in an artificial material to cover the native diseased vessel. The long incision is closed, and the excess tissue is discarded. In this case, there is no treatment to the native vessel; instead, an intimate prosthetic is placed bridging healthy tissue to healthy tissue. In an endovascular repair, an implant is introduced via a femoral access site and deployed within the lumen of the thoracic aneurysm. As in the open surgical procedure, the endovascular implant bridges between two healthy portions of the native aorta.

Both of these solutions are based on the premise that a reduction of pressure will reduce the risk of rupture in the native vessel. Degradation of the vessel, however, is not initiated by pressure, but exacerbated by pressure. Thus, in accordance with several embodiments, reduction of pressure alone does not provide a complete treatment of an aortic tear or dissection. Within the vessel wall, a cycle of inflammation may cause the connective tissue within the extracellular matrix to degrade. While collagen can be regenerated, elastin may be lost permanently. The body may compensate for the loss of elastin by producing additional collagen, but long-term this may not be sustainable.

Described herein are systems and methods for the treatment of an aortic tear or dissection which include the delivery of a stabilizing compound or agent, such as a compound including 1,2,3,4,6-Pentagalloyl Glucose (PGG) into the diseased portion of the aorta (e.g., thoracic aorta or other aortic regions), as well as within dissected portions of the aorta (e.g., thoracic aorta or other aortic regions).

Presented herein are several versions of delivery within the aorta (e.g., thoracic aorta or other aortic regions). In accordance with several embodiments, systems of devices and a therapeutic stabilizer compound described and illustrated herein provide aortic tissue stabilization by slowing or eliminating the progressive weakening of the aortic wall. Several embodiments are particularly advantageous because they include one, several, or all of the following benefits: (i) reduced hemodynamic pressure on already diseased aortic tissue, (ii) increased stabilization near the contact point of an endoprosthesis; (iii) reduction of the spread of diseased and weakened aortic tissue along the aortic wall, and/or (iv) increased stabilization of a false lumen of an aortic dissection or tear.

In some embodiments, the distal end of an infusion catheter is placed between an endoprosthesis and the diseased aortic wall, near the inlet flap of an aortic dissection, and stabilizer compound is infused. In some embodiments, the infusion catheter is placed within the false lumen of the aortic dissection and stabilizer compound is infused. In some embodiments, a weeping balloon for delivery of a stabilizer compound is coupled to the central passage of an endoprosthesis placed in an aortic aneurysm. In some embodiments, a flow diverting catheter is placed within a lumen of a thoracic aortic aneurysm, and stabilizer compound is infused. In some embodiments, an infusion catheter is placed near an inlet flap of an aortic tear or dissection and the infusion catheter is expanded to come into contact with the diseased aortic wall near the inlet flap.

In some embodiments, the infusion catheter includes one or more inflatable balloons. Such a balloon may comprise an elastic material forming an expandable membrane as is known in the art and may be configured to expand upon pressurization from an inflation fluid (for example, a gas or a liquid, such as saline). The balloon material may be biocompatible. In some embodiments, infusion catheters described herein comprise one or more perforations. In some embodiments of these infusion catheters, the infusion catheters are made with a porous covering instead of, or in addition to, having perforations.

In accordance with several embodiments, the stabilization treatment compound or agent advantageously reduces the rate of disease progression and prevents additional degradation of already diseased aortic tissue. The following are proposed methods and administration of stabilization treatment compound or agent for aortic tears and dissections (such as in a thoracic aortic region or other aortic regions).

Treatment of the Inlet Tear from the True Lumen:

In one embodiment, an infusion catheter is placed between a covered or uncovered endoprosthesis, which is designed to be expanded over the inlet flap, and the boundary of the true lumen (which may be aortic wall or a dissection flap). In some embodiments, the infusion catheter is placed first, while in other embodiments the undeployed stent is placed first. In some embodiments, expansion and placement of the stent results in the infusion catheter being placed in contact with the region of aortic tissue near the inlet flap. The apposition of the infusion catheter against aortic tissue may be limited to just the aortic region outside of the dissection flap, or it may include this undissected region along with the dissected region. In some embodiments, once apposed, a stabilization treatment compound is infused. In other embodiments, the infusion catheter is coated with a stabilizing treatment compound. This compound may be applied just to the undissected aortic wall, or to both the undissected aortic wall and the dissection flap. The stabilizing compound may comprise a contrast media agent, a stabilizing agent, and/or other excipients. The stabilizing agent can be a polyphenolic compound, like 1,2,3,4,6-Pentagalloyl Glucose (PGG). Once sufficient compound has been delivered, the infusion catheter may be removed. In some embodiments, the (covered or uncovered) stent is left in place after removal of the infusion catheter.

Methods of Treatment from the True Lumen Using an Infusion Catheter Comprising a Linear Catheter

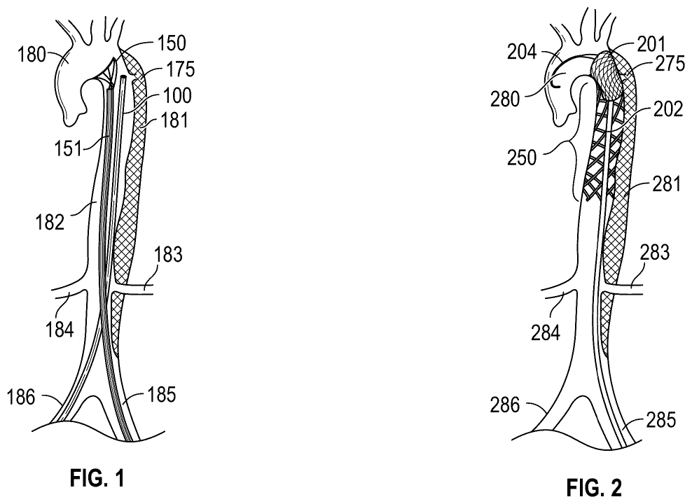

Described herein are methods of treating an aortic tear or dissection from the true lumen using an infusion catheter. The infusion catheter can take many different forms, such as those described herein. Infusion catheters shown in one figure may be used in connection with methods, devices or systems shown in other figures. In some embodiments, the infusion catheter is linear in shape. In the embodiment of FIG. 1, a guide catheter 151 is used for placing an endoprosthesis 150 (e.g., stent or other endovascular implant) near the inlet flap 175 of an aortic tear or dissection. An infusion catheter 100 is also placed near the inlet flap 175 to allow for treatment of the aortic tear or dissection. In some embodiments, the infusion catheter 100 is placed between an outer surface of the endoprosthesis 150 and the aortic wall. The partial cross-sectional view of FIG. 1 depicts an ascending aorta 180, a false lumen 181, a true lumen 182, a left renal artery 183, a right renal artery 184, a left common iliac artery 185, and a right common iliac artery 186. In some embodiments, the infusion catheter 100 comprises a linear tube with perforations or openings in the distal portion where the endoprosthesis 150 is to be placed. The infusion catheter 100 may be shaped in some embodiments in a manner specifically designed to facilitate its passage through or behind the endoprosthesis 150. The infusion catheter 100 may extend a short distance beyond the ends of the endoprosthesis 150 (e.g., a stent or implant) so that the aortic tissue immediately beyond or adjacent the stent also receives the stabilizer or stabilizing compound or agent.

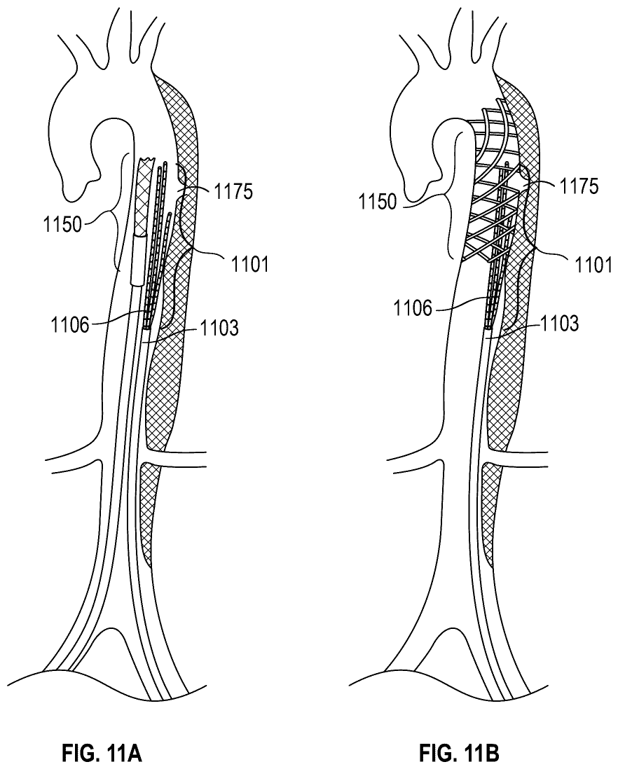

Some embodiments relate to methods of delivering stabilization treatment (e.g., stabilization compound or agent) via an infusion catheter comprising one or more linear catheters (e.g., one, two, three, four, five, or more than five catheters). As shown in FIG. 11A, an infusion catheter 1100 with a substantially linear shape may be placed between a thoracic endovascular aneurysm repair (TEVAR) implant 1150 and the vessel wall. In some embodiments, the infusion catheter 1100 includes one linear catheter 1101 connected to a single proximal catheter 1103 with fluid communication to a proximal handle. In some embodiments, the infusion catheter 1100 includes two to five linear catheters 1101 connected to a single proximal catheter 1103 with fluid communication to a proximal handle. In the embodiment of FIGS. 11A and 11B, the infusion catheter 1100 includes three linear catheters 1101 connected to a single proximal catheter 1103 with fluid communication to a proximal handle. In some embodiments, the one or more linear catheters 1101 are covered with a soft elastomeric material. In some embodiments, the one or more linear catheters 1101 comprise one or more perforations or openings 1106. In the embodiment shown in FIGS. 11A-11B, the one or more linear catheters 1101 comprise perforations 1106 along the distal portion of the linear catheter 1101. The perforations 1106 may surround the circumference of the distal portion or may only be located on a portion of the circumference (e.g., on one half of the circumference).

In some embodiments, and as shown in FIG. 11A, the distal portion of the infusion catheter 1100 is placed between an outer surface of the undeployed TEVAR implant 1150 and the diseased aortic tissue, prior to the deployment of the TEVAR implant 1150. In some embodiments, as the TEVAR implant 1150 is deployed and exposed, the infusion catheter 1100 is pushed to the aortic wall along with the TEVAR implant 1150. In some embodiments, and as shown in FIG. 11B, the infusion catheter is apposed to the aortic wall near the inlet flap 1175. In some embodiments, once apposed to the wall, a stabilization treatment solution (such as a solution containing PGG) is infused along the one or more linear catheters 1101 from the proximal handle and distributed amongst the one or more perforated linear catheters 1101 between the TEVAR implant 1150 and the vascular wall.

In some embodiments, the infusion catheter may be in the form of many other different configurations and shapes including, but not limited to, a mesh or matrix of fluid communicating materials. In another embodiment, instead of a liquid stabilizing treatment compound, the stabilizer (e.g., stabilizing agent) may comprise a coating on the endoprosthesis (e.g., stent, covered or uncovered) which is placed over the inlet flap. In some embodiments, over time, the active stabilizing agent is then eluted into the aortic tissue (wall and/or dissection flap) over a period of time (for example, 2 days to 21 days). In some embodiments, only that portion of the endoprosthesis intended to be deployed beyond the dissection flap is coated with the stabilizer (e.g., stabilizing agent), such that stabilizer contact with the dissection flap is avoided. In other embodiments, the entire endoprosthesis (covered or uncovered) is coated with the stabilizer (e.g., stabilizing agent) so that all adjacent aortic tissue is treated by the stabilizing compound.

Methods of Treatment from the True Lumen Using an Infusion Catheter Comprising a Spiral Catheter

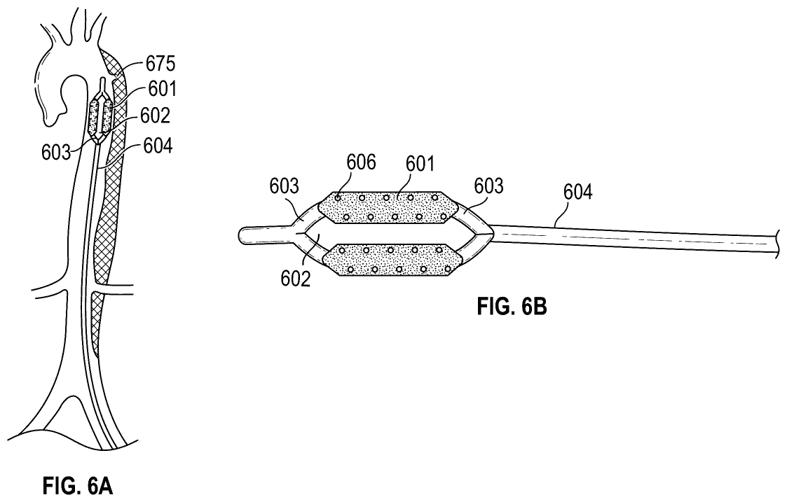

In some embodiments, the infusion catheter comprises a curvature. In the embodiment of FIG. 5, an infusion catheter 500 with an internal wire stylet 501 has been placed within the true lumen of the aorta near an inlet flap 575 and has been manipulated to obtain the shape of a spiral 502. The partial cross-sectional view of FIG. 5 depicts an ascending aorta 580, a false lumen, a true lumen, a left renal artery 583, a right renal artery 584, a left common iliac artery 585, and a right common iliac artery 586. In some embodiments, the curvature may comprise a spiral 502. In some embodiments, the infusion catheter 500 comprises a shape memory material, such as polymer, plastic, or metal, designed to make at least one revolution within the true lumen. In some embodiments, the infusion catheter 500 further comprises an actuator. In some embodiments, the infusion catheter 500 comprises an inner wire stylet 501. In some embodiments, the infusion catheter 500 may be manipulated from the proximal end to the distal end to achieve a desired shape, such as a spiral 502 or corkscrew or pigtail shape. In some embodiments, the infusion catheter 500 may be shortened to accentuate its curvature to contact aortic tissue or lengthened to achieve a straighter configuration. In the embodiment of FIG. 5, the infusion catheter 500 contacts the walls of the aorta once it has been actuated to form a spiral 502 shape.

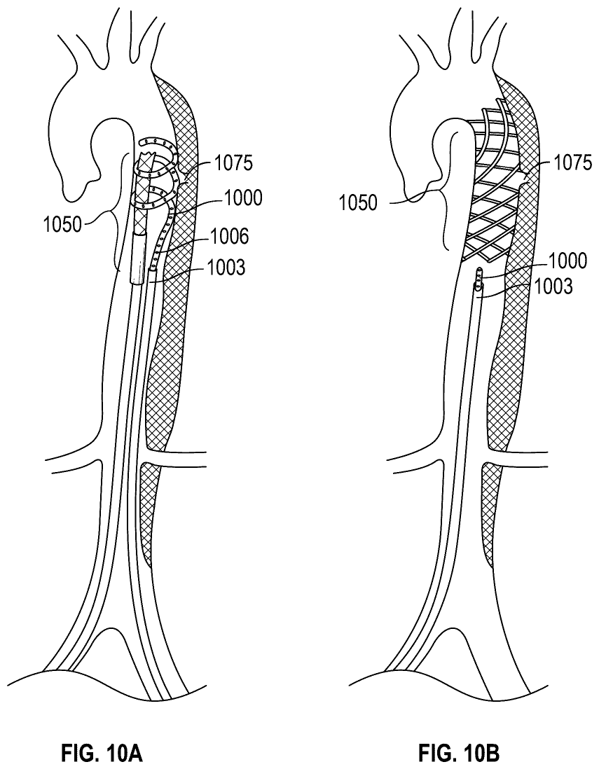

In the embodiment of FIG. 10A, an infusion catheter 1000 actuated to have a spiral shape is placed near an inlet flap 1075 prior to placement of a thoracic endovascular aneurysm repair (TEVAR) implant 1050. After the infusion catheter 1000 is placed, a stabilization treatment compound is delivered to the target tissue.

In some embodiments, the infusion catheter 1000 has an inner rigid member, such as a nitinol wire that maintains the shape of the infusion catheter 1000. In some embodiments, the shape is encapsulated in an encapsulating catheter tube 1003. In some embodiments, once the infusion catheter 1000 has been advanced to the desired location (such as near the inlet flap 1075), the encapsulating catheter tube 1003 is retracted back while feeding the rigid member of the infusion catheter 1000 into the vasculature. In some embodiments, the infusion catheter 1000 is pre-shaped to a spiral configuration. In some embodiments, the pitch of deployment in the vasculature is influenced by the pre-shape and the rate at which it is advanced into the vessel.

In some embodiments, for delivery of the stabilizing treatment compound, infusion catheter 1000 is covered in an elastomeric material that includes one or more perforations or pores 1006. In some embodiments, and without being bound by theory, the rate of perfusion of the stabilizing compound may be influenced by the size of the perforation or pore diameter, quantity, and/or viscosity of the stabilizing treatment compound. For example, a perforation diameter between 0.0015 and 0.005 inches may allow an equalized pressure along the elastomeric length prior to full infusion. In some embodiments, infusion will preferentially fill the entire length of the elastomeric material prior to exiting the perforations 1006. In some embodiments, the elastomeric material is a soft, non-compliant plastic or polymer. In some embodiments, the shape is conformable to a spiral stylet.

In some embodiments, the system is placed, and non-compliant plastic, metal, or polymer maintains its spiral position and allows for infusion of stabilizing treatment compound without resistance. In some embodiments, subsequent to the infusion of the stabilizing treatment compound through the infusion catheter 1000, the TEVAR implant 1050 is advanced within the loops of the spiral of the infusion catheter 1000, as is shown in FIG. 10A. In some embodiments, the TEVAR implant 1050 is advanced once the infusion has stopped. In some embodiments, the TEVAR implant 1050 is advanced through the spirals of the infusion catheter 1000 after the infusion has begun but before infusion has finished.

In some embodiments, the spiral stylet is removed prior to the deployment of the TEVAR implant 1050 to the aortic wall. In some embodiments, the spiral stylet is removed after the deployment of the TEVAR implant 1050 to the aortic wall. As shown in the embodiment of FIG. 10B, the infusion catheter 1000 may be removed and may be covered with an encapsulating catheter 1003. In some embodiments, the infusion catheter 1000 is retracted into an encapsulating catheter 1003 by pulling on the proximal end of the infusion catheter 1000 or by otherwise manipulating the proximal end of the infusion catheter 1000. In the embodiment of FIG. 10B, the TEVAR implant 1050 has been deployed near the inlet flap 1075 and the infusion catheter 1000 is removed.

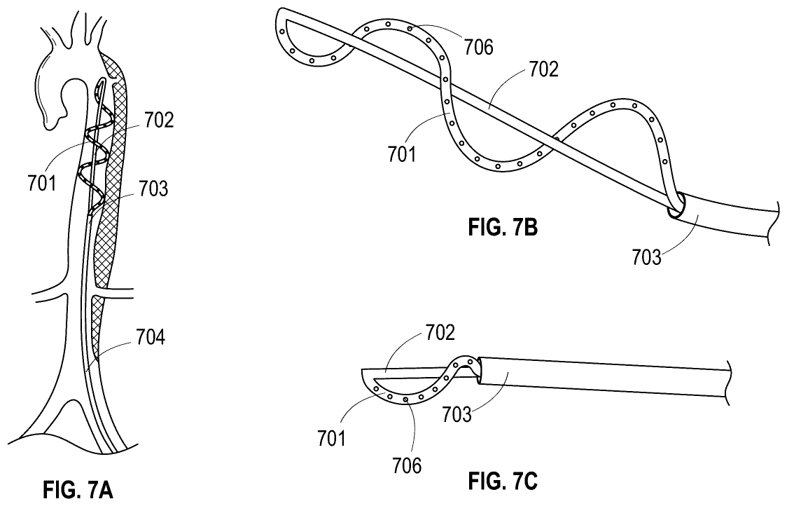

In some embodiments, an infusion catheter 700 which includes at least two tubes is deployed within a true lumen of a thoracic aortic aneurysm to treat an aortic tear or dissection. In some embodiments, the infusion catheter 700 comprises an actuating catheter tube 702, and a fluid communication tube 701 configured to be actuated to form a spiral around the actuating catheter tube 702. In some embodiments, a first fluid communication tube 701 allows for fluid communication from the proximal end of the infusion catheter 700. In some embodiments, the other tube is an actuating catheter tube 702 which allows actuation which causes the fluid communication tube 701 to take the shape of a spiral. In the embodiment of FIG. 7A, an infusion catheter 700 with a first fluid communication tube 701 actuated to form a spiral along the thoracic aortic wall, and second actuating catheter tube 702, has been deployed in a true lumen of a diseased thoracic aorta or other aortic region. In the embodiment of FIG. 7A, the fluid communication tube 701 is actuated to form a spiral along the thoracic aortic wall, and portions of the fluid communication tube 701 are in contact with the aortic wall.

A further view of the infusion catheter 700 is depicted in FIG. 7B. In some embodiments, the fluid communication tube 701 is comprised of a rigid material, for example a nitinol wire, secured within the tube 701. In some embodiments, around the rigid material or wire is a balloon-like material which is sealed around the first catheter fluid communication tube 701, such that fluid infused from the proximal end of the first catheter tube 704 flows around the rigid wire and into the balloon like material. In some embodiments, the balloon-like material may be elastomeric. In some embodiments, the balloon-like material is soft and easily conformable. In some embodiments, the shape of the balloon-like material can be tubular or made flat by fusing spots or lines between the top and bottom portions.

In some embodiments, the second actuating catheter tube 702 slides within the first fluid communication tube 701. In some embodiments, the proximal end of the second actuating catheter tube 702 is made to be guidewire compatible and to maintain hemostasis. In some embodiments, the distal end of the second actuating catheter tube 702 is connected to the rigid material or wire and the balloon-like material of the first fluid communication tube 701. In some embodiments, while all are terminated together, there is still a taper and a lumen at the distal end for guidewire compatibility.

In some embodiments, when the second actuating catheter tube 702 is in a distal position, the rigid wire and balloon-like material of the first fluid communication tube 701 are in a low-profile configuration for insertion into the vessel via a guidewire and introducer sheath 703. In some embodiments, the wire is wrapped around the second actuating catheter tube 702 at least once. In some embodiments, when in a proximal configuration, the second actuating catheter tube 702 allows for the rigid wire of the first fluid communication tube 701 to unwind outward and for the balloon-like material to contact the vessel wall.

In some embodiments, a stabilizing treatment compound or agent such as PGG is delivered through the balloon-like material which has one or more perforations, holes, or pores 706. In some embodiments, the perforation hole size, quantity, and stabilizing treatment fluid viscosity control the fluid infusion behavior. In some embodiments, the perforation holes are between 0.0015 and 0.005 inches. In some embodiments, a radiopaque stabilizing treatment solution (such as one containing PGG) is infused into the first fluid communication tube 701 via a fluid lumen. In some embodiments, the liquid stabilizing treatment compound inflates the entire soft balloon-like material. In some embodiments, once inflated, additional infusion allows for the stabilizing treatment solution to leak slowly out of the perforations 706 and near the target tissue. In some embodiments, and without being bound by theory, blood perfusion would have minimal resistance based on the cross-sectional profile of the infusion catheter 700. In some embodiments, infusion can be between 2 and 15 minutes (e.g., between 2 minutes and 5 minutes, between 3 minutes and 8 minutes, between 2 minutes and 10 minutes, between 5 minutes and 10 minutes, between 5 minutes and 15 minutes, between 6 minutes and 12 minutes, overlapping ranges thereof, or any value within the recited ranges) to provide a therapeutic dose.

FIG. 7C is a side view of an embodiment of the infusion catheter of FIG. 7B, where the first fluid communication tube 701 and the second actuating catheter tube 702 are partially enclosed within an introducer sheath 703. In some embodiments, once complete, residual stabilization treatment solution is evacuated, the second actuating catheter tube 702 is moved to a distal position to lower the profile, and the entire infusion catheter 700 system is withdrawn over a guidewire.

In another embodiment, the second actuating catheter tube 702 is slid distally a fixed distance that matches the treatment length. In such an embodiment, the rigid material or wire may be covered in an elastomeric material and the first fluid communication tube 701 may be advanced such that the rigid wire material forms a spiral. In some embodiments, the distal end of the spiral starts with a small inner diameter, but with additional length of rigid wire pushed distally, the diameter expands outward. The resulting shape would be a tube with perfusion in the middle and the structure of the tube consisting mostly of the elastomeric material and perforations 706 substantially on the circumferential portions of the material for inflation and perfusion of stabilization treatment solution to the target tissue.

Methods of Treatment from the True Lumen Using Infusion Catheters Comprising a Balloon

Provided herein are methods of treating an aortic tear or dissection that include delivering a stabilizing treatment compound via an infusion catheter comprising a balloon, such as a perforated balloon. In some embodiments, a balloon (for example, a toroidal balloon) with a large central passage, forming a flow path from the proximal end to the distal end, is placed outside the stent prior to deployment. In some embodiments, a balloon is placed between an undeployed stent and diseased aortic tissue. As the stent is deployed, the soft balloon material (inner wall and outer wall) is pushed against the aortic tissue (either wall or dissection flap). The stabilizer compound is then infused into the balloon, and weeps onto the aortic tissue through discrete perforations or material porosity in the outer wall of the balloon. In some embodiments, the balloon is inflated and infuses stabilizing treatment compound onto diseased aortic tissue and is then removed prior to placement and deployment of a stent.

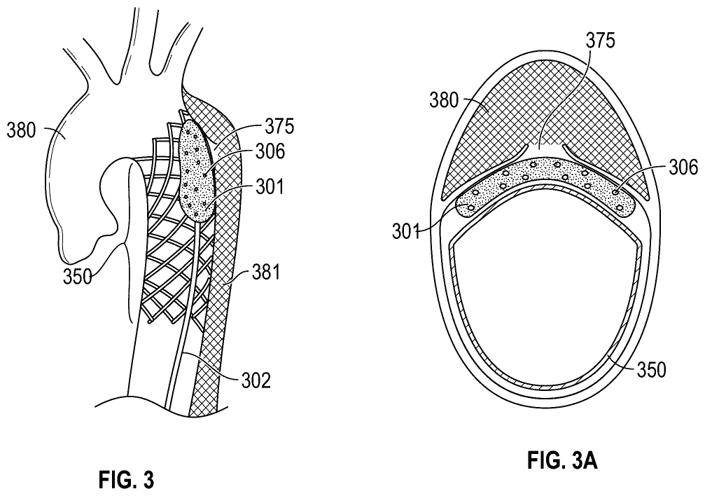

In some embodiments, the methods comprise inserting an infusion catheter comprising a balloon with a central passage coupled to an endoprosthesis. The partial cross-sectional view of FIG. 2 depicts an ascending aorta 280, a false lumen 281, a true lumen, a left renal artery 283, a right renal artery 284, a left common iliac artery 285, and a right common iliac artery 286. In the embodiment of FIG. 2, an infusion catheter 200 comprising a balloon 201 with a central passage coupled to a stent 250 has been deployed near the inlet flap of the aortic tear or dissection. Such a balloon 201 may be constructed from any typical materials used to construct intravascular balloons. In a preferred embodiment, the balloon 201 is constructed from a noncompliant or semi-compliant material and is sized such that the fully expanded balloon diameter is larger than the aortic diameter to be treated so that the outer wall of the balloon 201 may be pushed by the stent 250 into contact with the aortic tissue (wall or dissection flap 275). In some embodiments, a stabilizing treatment compound is delivered to the balloon via a balloon catheter 202. In some embodiments, the balloon 201 may comprise perforations to allow infusion of a stabilizing treatment compound onto a diseased aortic wall, including near the inlet flap 275.

In some embodiments, the balloon 201 is preloaded into a coaxial restraining sleeve through which the stent 250 is placed prior to insertion in the body. The loaded stent 250 and balloon 201 may be placed in the proper position near the inlet flap 275. Once the stent 250 and balloon 201 are in position, the restraining sleeve is removed (retracted or released), thereby allowing the expanding stent 250 to push the balloon 201 into position against the aortic wall. The system of FIG. 2 may further comprise a wire stylet or guide wire 204. In some embodiments, after stabilizer delivery is complete, the balloon 201 may be deflated and then pulled out from around the deployed stent 250, after which the balloon 201 may be recaptured into a sheath for removal from the vasculature.

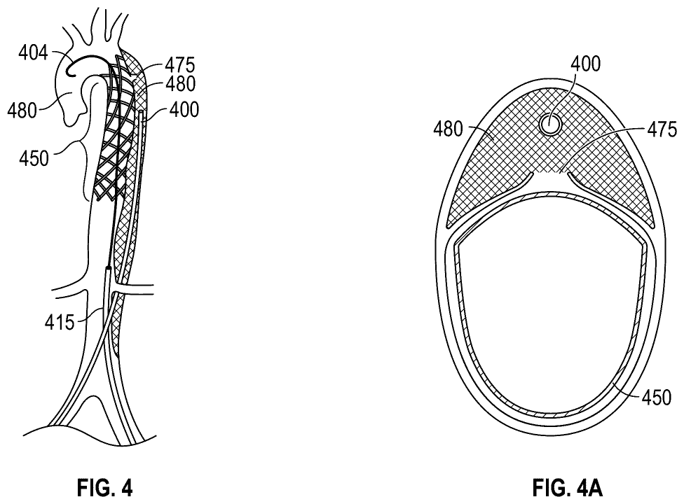

In some embodiments, a balloon is placed between an undeployed stent and diseased aortic tissue. The side partial cross-sectional view of FIG. 3 depicts an ascending aorta 380, a false lumen 381, a true lumen, and an inlet flap 375 of a thoracic aortic aneurysm. In the embodiment of FIG. 3, an endoprosthesis 350 and an infusion catheter 300 comprising a balloon 301 and a balloon catheter 302 have been placed near the inlet flap 375 of the aortic tear or dissection. In some embodiments, the balloon 301 is placed between an undeployed endoprosthesis 350 and the diseased aortic tissue. In some embodiments, the endoprosthesis 350 is then deployed. In some embodiments, deployment of the endoprosthesis 350 pushes the balloon 301 into contact with aortic wall. In some embodiments, the balloon 301 comprises perforations 306. In some embodiments, a stabilizing compound is infused through the balloon catheter 302, into the balloon 301, and onto the aortic tissue via the perforations 306. Thus, infusion catheter 300 may infuse a stabilization treatment compound onto the aortic tissue surrounding the inlet flap 375.

FIG. 3A is a cross-sectional view of FIG. 3 along an axis perpendicular to a longitudinal axis of the aorta. In the embodiment of FIG. 3A, the endoprosthesis 350 has been deployed such that the balloon 301 is pressed between the endoprosthesis 350 and the diseased aortic wall near the inlet flap 375. The balloon 301 may be constructed from any typical materials used to construct intravascular balloons. In some embodiments, the balloon 301 is constructed from a noncompliant or semi-compliant material. In some embodiments, the balloon 301 is preloaded into a coaxial restraining sleeve through which the endoprosthesis 350 is placed prior to insertion in the body. In other embodiments, the balloon 301 is placed beside the undeployed endoprosthesis 350 independently, once within the body. In such an embodiment, either the endoprosthesis 350 or the balloon 301 may be placed first. In some embodiments, once the endoprosthesis 350 and balloon 301 are in position, a restraining sleeve around the endoprosthesis 350 is removed (retracted or released), thereby allowing the expanding endoprosthesis 350 to push the balloon 301 into position against the aortic wall. After stabilizer delivery is complete, the balloon 301 may be deflated and then pulled out from under the deployed endoprosthesis 350, after which the balloon 301 may be recaptured into a sheath for removal from the vasculature.

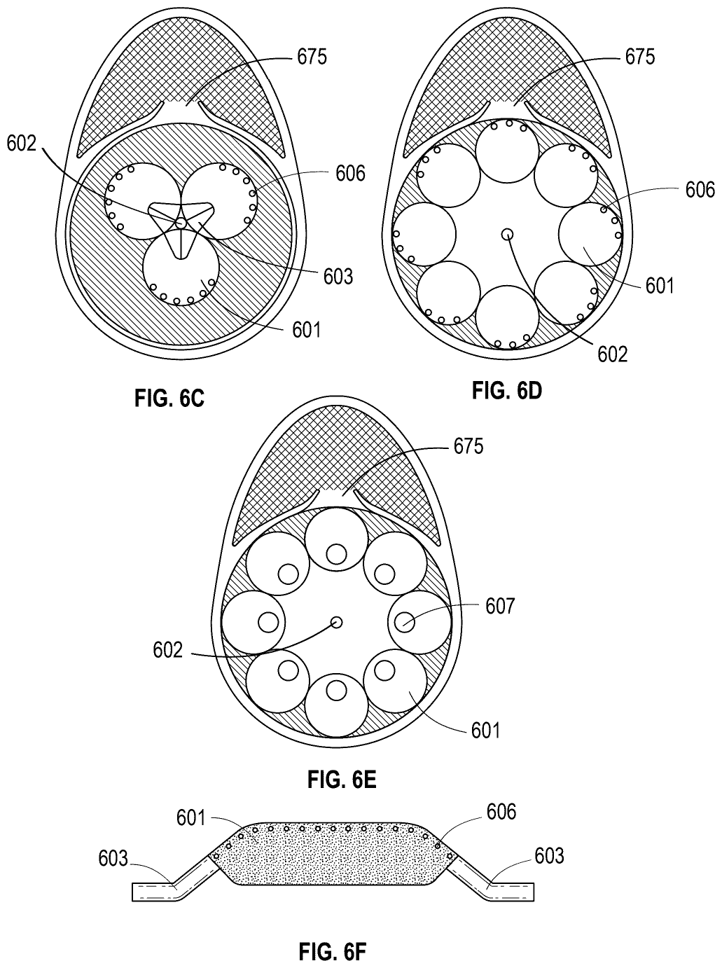

FIG. 6A depicts an embodiment where an infusion catheter 600 comprising two or more elongated balloons 601 is placed within the true lumen near the inlet flap 675. In such an embodiment, the two or more elongated balloons 601 may be inflated within the vessel while allowing perfusion to flow through the interstitial space between the balloons. Depicted in FIGS. 6A-6C is an infusion catheter 600 with a 3-balloon system. A side view of an infusion catheter 600 comprising 3 or more elongated balloons is shown in FIG. 6B.

As shown in FIGS. 6A-6B, and particularly in FIGS. 6C and 6F, in some embodiments, each balloon 601 is bound to a tubular lumen 602 both on the proximal and distal ends of the balloon 601, such as with attachment lumens 603. In some embodiments, the attachment lumens 603 provide fluid communication with the tubular lumen 602. In some embodiments, each elongated balloon 601 is connected to two attachment lumens 603, and only one attachment lumen 603 of the two attachment lumens 603 provides fluid communication with the tubular lumen 602. In some embodiments, the distal ends of the elongated balloons 601 are all terminated together (such as through independent attachment lumens 603) into a single main-body catheter 604. In some embodiments, the proximal ends of the elongated balloons 601 are bound to and connected to (including, in some embodiments, a fluid communication) the main-body catheter 604 through one or two independent attachment lumens 603. In some embodiments, the main-body catheter 604 has at least 2 lumens; one for guidewire compatibility and the other lumen(s) for inflation of the balloons 601. In some embodiments, the proximal end of the main-body catheter 604 allows for guidewire compatibility and independent or simultaneous inflation of the balloons 601.

FIG. 6C depicts of a cross sectional view of FIG. 6A along an axis perpendicular to a longitudinal axis of the aorta. In some embodiments, the outer walls of the elongated balloons 601 comprise one or more pores or perforations 606. In some embodiments, one or more of the balloons 601 are placed within the true lumen near an inlet flap 675 of an aortic tear or dissection. FIG. 6C depicts an embodiment where the attachment lumens 603 of the distal ends of the elongated balloons 601 are bound and terminate together.

FIG. 6D depicts a cross section view along an axis perpendicular to a longitudinal axis of the aorta with an example of an infusion catheter 600 including eight elongated balloons 601. In some embodiments, the outer walls of the elongated balloons 601 comprise one or more pores or perforations 606. In some embodiments, one or more of the balloons 601 are placed within the true lumen near an inlet flap 675 of an aortic tear or dissection. In the embodiment of FIG. 6D, the elongated balloons are inflated to contact the vessel.

FIG. 6E depicts a cross section view along an axis perpendicular to a longitudinal axis of the aorta with an example of an infusion catheter 600 including eight elongated balloons 601. In some embodiments, a perfusion tube 607 is disposed within one or more elongated balloons 601. In some embodiments, the outer walls of the elongated balloons 601 comprise one or more pores or perforations 606. In some embodiments, one or more of the balloons 601 are placed within the true lumen near an inlet flap 675 of an aortic tear or dissection. In the embodiment of FIG. 6E, the elongated balloons are inflated to contact the vessel.

FIG. 6F depicts a side view of a balloon 601 of an infusion catheter 600. In some embodiments, the balloon 601 is 50 mm-90 mm in length. In some embodiments, the proximal end of the elongated balloon 601 connects to the main-body catheter 604 through an independent attachment lumen 603, and the distal end of the elongated balloon 601 connects to the main-body catheter 604 through an independent attachment lumen 603. In the embodiment of FIG. 6F, there are perforations or pores 606 on the circumferential portion of the balloon 601.

In some embodiments, the one or more elongated balloons 601 deliver a stabilizing treatment compound to the aortic wall. In some embodiments, the stabilizing treatment is delivered as a coating on the circumferential portions of the balloons 601. Without being bound by theory, in some embodiments, a primary coat of stabilizing treatment compound would create a matrix which would entrap molecules of the stabilizing treatment compound, which would be released slowly when exposed into an aqueous environment like a blood vessel. The matrix can be designed to be hydrophilic or hydrophobic, and to be activated by blood, water, or air. In some embodiments, the one or more balloons 601 are loaded via a dipping process prior to treatment. Once apposed to the wall, delivery of a therapeutic dosage of stabilizing treatment compound can range between 2 minutes and 15 minutes. In some embodiments, the stabilizing treatment compound is PGG. The stabilizing treatment compound may be provided in a liquid solution.

In some embodiments, the one or more elongated balloons 601 deliver a stabilizing treatment as a liquid. In some embodiments, stabilizing treatment compound is infused into the two or more balloons 601, such as via a main-body catheter 604. In some embodiments, tiny perforations or pores 606 are made into the circumferential portions of the balloons 601. The size, quantity of holes, and viscosity of the fluid can influence the inflation behaviors of the balloons 601. In some embodiments, the infusion of stabilizing treatment into the balloons 601 displaces blood while inflating while not losing any of the stabilizing treatment solutions through the perforations 606, until inflation is complete. In some embodiments, upon apposition of the circumferential portions of the balloons 601 to the wall, additional infusion would encounter enough resistance that preferential movement of fluid would be through the perforations 606 rather than continued inflation of the balloons 601. In some embodiments, perfusion is achieved through interstitial space created by the balloons. In some embodiments, while the pressure is increased due to the restriction, forced deflation of the balloons 601 is minimized by not placing perforations 606 on the distal end of the balloon 601 taper. In some embodiments, duration of wall apposed infusion ranges between 2 minutes and 15 minutes.

In some embodiments, once delivery is complete, the balloons 601 are deflated, and the infusion catheter 600 is removed via a guidewire (such as a guidewire disposed within a lumen of the main-body catheter 604).

Treatment of the Inlet Tear from the False Lumen:

In another aspect, an infusion catheter is placed in the false lumen through one of the re-entry tears in the aorta. The re-entry tear may be located near the renal arteries. In some embodiments, a (covered or uncovered) stent is placed in the true lumen. The infusion catheter may be placed first, or the endoprosthesis may be placed first. In some embodiments, with the infusion catheter in place, the stabilization treatment compound is infused. The stabilization treatment compound may comprise a contrast media agent, a stabilizing agent, and other excipients. The stabilizing agent may comprise a polyphenolic compound, like 1,2,3,4,6-Pentagalloyl Glucose (PGG). In some embodiments, once sufficient compound has been delivered, any remaining stabilization treatment compound may be aspirated, and the infusion catheter may be removed. This treatment may be combined with the treatment description above, so that aortic regions above and below the dissection may also be treated with the stabilizer, with or without the deployment of the stent in these undissected regions.

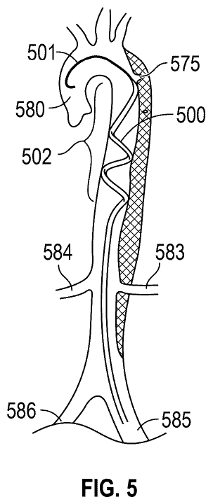

The infusion catheter can take many forms. For example, in some embodiments, the infusion catheter comprises a substantially linear tube with perforations in the distal sections, beyond the re-entry into the false lumen. The side partial cross-sectional view of FIG. 4 depicts an ascending aorta 480, a false lumen 481, a true lumen, and an inlet flap 475 of a thoracic aortic aneurysm with an aortic dissection. In the embodiment of FIG. 4, an infusion catheter 400 has been placed in the false lumen 481 through one of the re-entry tears of the aortic dissection, an endoprosthesis 450 has been deployed near the inlet flap 475. In some embodiments, the infusion catheter 400 is placed in the false lumen 481 through a retrograde flap before the endoprosthesis 450 is introduced into the true lumen. In some embodiments, the infusion catheter 400 is placed in the false lumen 481 through a retrograde flap before the endoprosthesis 450 is deployed in the true lumen. In some embodiments, the infusion catheter 400 is placed in the false lumen 481 through a retrograde flap after the endoprosthesis 450 is deployed in the true lumen.

The distal end of the infusion catheter 400 may comprise one or more perforations or openings. Stabilizing treatment may be infused into the infusion catheter 400 and into the false lumen 481 through the one or more perforations. In some embodiments, the infusion catheter 400 is removed after delivery of the stabilizing treatment compound. In some embodiments, the infusion catheter 400 is removed after deployment of the endoprosthesis 450.

In some embodiments, and as seen in FIG. 4, a second catheter 415 with an internal wire stylet or guide wire 404 has been placed in the true lumen of the aorta. In some embodiments, the second catheter 415 is a catheter used to deploy the endoprosthesis 450. In other embodiments, the second catheter 415 is a second infusion catheter 400 used to deliver a stabilizing treatment compound to the true lumen, as has been described herein. FIG. 4A depicts a cross-sectional view of FIG. 4 along an axis perpendicular to a longitudinal axis of the aorta and shows the infusion catheter 400 placed within the false lumen 481, while the endoprosthesis 450 has been deployed in the true lumen near the inlet flap 475.

In another embodiment, a secondary tube may be slid axially over the infusion catheter at or near the re-entry outlet to occlude more proximal perforations on the catheter; thereby allowing infusate to reside within the false lumen. In some embodiments, with the optional addition of an actuator, the infusion catheter may also be manipulated from the proximal end to the distal end to achieve its shape (for example, a spiral). In some embodiments, the infusion catheter may be shortened to accentuate its curvature in order to contact aortic tissue or lengthened to achieve a straighter configuration. In some embodiments, the infusion catheter is in the form of many different configurations and shapes including, but not limited to, a mesh or matrix of fluid communicating materials.

For both treatment via the inlet tear or retrograde via the false lumen, the infusion catheter may comprise a perforated balloon instead of a perforated catheter shaft. The balloon may comprise a variety of materials including, but not limited to, a soft elastomeric material which is able to conform to any shape within the false lumen. Inflation with the stabilizing treatment compound would preferentially inflate the balloon because of the reduced pressure in the false lumen following deployment of an endoprosthesis at the inlet tear. Upon displacement of the blood from the false lumen, increased infusion of the stabilizing treatment compound would result in a pressure release and stabilizer weeping out of the perforations, thereby creating a liquid film layer on the outside of the balloon. In some embodiments, once a therapeutic amount of liquid has been delivered, the remaining amount of stabilizing treatment compound within the balloon may be evacuated and the system may be retrieved. Deployment and retrieval of the balloon may be direct, or the balloon may be sheathed to reduce the profile through the re-entry tear.

### Methods of Treatment Using Flow Diversion:

In an embodiment, a flow diverting catheter is placed within the lumen. The flow diversion provides perfusion to the aortic arch and/or the descending aorta. The flow diversion may create at least a 10 mm channel for blood flow. The pressure (and flow) near the targeted aneurysmal tissue would be significantly lower. Either via the flow diverting catheter, or via another catheter, stabilizing treatment compound is infused. The stabilizing treatment compound comprises a contrast media agent, a stabilizing compound, and other excipients. The stabilizing treatment compound may comprise a polyphenolic compound, like 1,2,3,4,6-Pentagalloyl Glucose (PGG). Once fully delivered, the excess stabilizing treatment may be aspirated.

The flow diverting catheter may optionally include an occlusive element on either its proximal or distal end, or both. The flow diverting catheter may also optionally include an additional occlusive element after the flow diverting channel which may be positioned in a healthy portion of the aorta.

In one embodiment of the first occlusive element, the first occlusive element comprises a balloon. In another embodiment, the first occlusive element comprises a sturdy ring covered with a flexible but pre-shaped material. The shape of the material may comprise a slow tapered cone with an open cylindrical outlet (much like a windsock). After the ring is apposed, blood would flow through the funnel (windsock) and exit the proximal end. While flexible, the shape is pre-fixed and produces a reduced pressure/flow region between the material and the aneurysmal wall. Through an integrated, or separate catheter, stabilizing treatment compound is infused into this reduced pressure annular space and the stabilizing agent allowed to penetrate the aneurysmal wall. In another embodiment, the flow diverting catheter comprises a proximal or second occlusive element to fully isolate the aneurysmal wall. The stabilizer treatment compound may also be infused in the annular, lower pressured, space between the material and the aneurysmal wall. In both embodiments, once sufficient volume is delivered, the remaining stabilizer treatment compound may be aspirated. Once aspirated, both rings may be removed, and normal flow restored.

In one embodiment, the flow diverting catheter comprises a balloon-like structure for one or both occlusive elements. A material or structured element may be placed between the occlusive balloon elements to form the perfusion channel. Axial support for the balloon may be supplied by an inflatable network of channels that create a supportive truss-like structure or may be created by a buckle-resistant mechanical support such as a braid reinforced catheter tube or similar technology.

In another embodiment, longitudinal support for the flow diverting structure may be provided by having the flow diverting structurally coaxial with and outside of a supporting, buckle-resistant mechanical structure. The flow diverting structure may be suspended from the tip of the support structure, much like a windsock from a pole; the support structure serves to resist the large hemodynamic forces that would otherwise cause the flow diverting structure to migrate distally away from the region of interest. The flow diverting structure may be initially compressed in a sheath that is coaxially external to the mechanical support structure, may be deployed by retraction of this sheath, and may be recaptured after stabilizer delivery by readvancement of this sheath for smooth, atraumatic removal from the vasculature. In another embodiment, deployment of the flow diverting structure may be controlled by compressing wires or sutures wrapped or attached to the outside such that when the wires/sutures are tensioned, the flow diverter collapses onto the mechanical support structure and when the wires/sutures are released, the spring-loaded nature of the flow diverter causes it to expand outward from the mechanical support structure and into contact with the aortic wall.

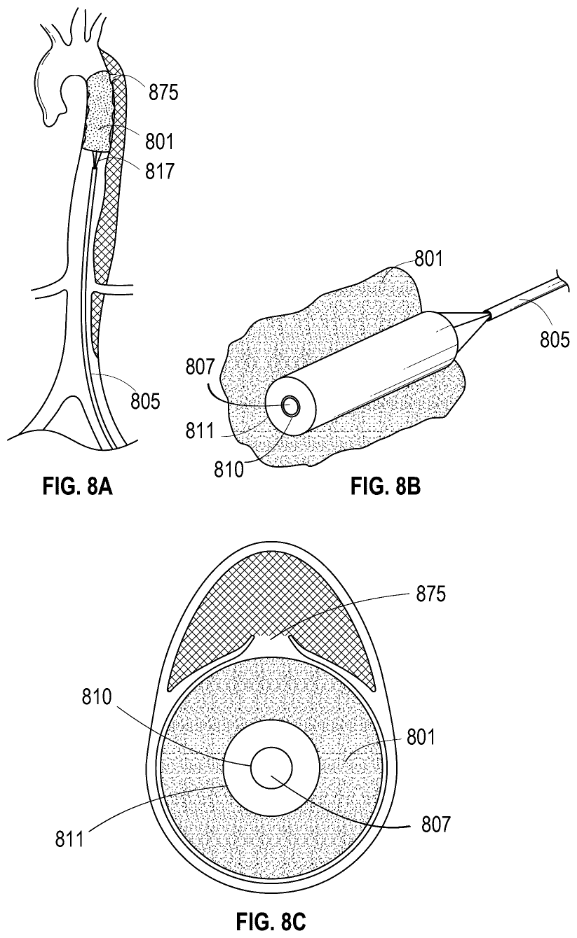

FIG. 8A depicts a side partial cross-sectional view of an aorta with an aortic tear or dissection, where an infusion catheter 800 delivers a stabilizing compound via a perforated balloon 801. FIG. 8B depicts an oblique partial cutaway view of the embodiment of FIG. 8A of the infusion catheter 800. As shown FIG. 8B, a perfusion lumen 807 is created while a stabilizing compound such as PGG is delivered peripherally through perforations 806 on the circumferential portions of the balloon 801.

In some embodiments, a proximal catheter 903 (not shown in FIGS. 8A-8C) is used to encapsulate the perfusion lumen 807 and outer balloon 801 covering. In one embodiment, the perfusion lumen 807 is created by an inner scaffold 810. In some embodiments, the inner scaffold 810 comprises a woven material, mesh-like material, multiple woven material tubes, spiral wound wire like nitinol, or multiple straight wires that run the length of the inner inflation tube. In some embodiments, the inner scaffold 810 collapses into the proximal catheter 903 when pulled proximally. In the embodiment of FIGS. 8A-8B, the proximal end of inner scaffold 810 and/or outer scaffold 811 is attached to the second catheter 805, by one or more strings 817 (e.g., “parachute” strings). In some embodiments, pulling on the proximal of the end of the second catheter 805 causes the inner scaffold 810 and/or outer scaffold 811 to collapse down as it is pulled proximally for removal of the infusion catheter 800 from the vessel. In some embodiments, when pulled from the distal ends, the inner scaffold 810 and outer scaffold 811 expand outward.

In some embodiments, the outer covering is a tubular balloon 801 with circumferential perforations or pores 806. In some embodiments, the first proximal catheter 903 would have a second catheter 805 that actuates within an encapsulation lumen, and which maintains fluid communication at the proximal end. In some embodiments, the second catheter 805 has a distal end which is in fluid communication with the balloon 801. In some embodiments, the balloon 801 is constructed as a dual layer. In some embodiments, four sheets of balloon material are fused such that the inner two-layers form a tube from the distal to proximal end, while the outer two layers are inflated around the tube. In some embodiments, the second catheter 805 is in fluid communication with the lumen formed by the top and bottom layer (outside) and inner layers (inside).

In some embodiments, the inner scaffold 810 maintains the inner layers open for blood perfusion. In some embodiments, the balloon 801 would be perforated on the outside circumferential portion of the balloons 801. In some embodiments, and without being bound by theory, fluid flow characteristics would be influenced by the perforation 806 hole diameter, quantity, and fluid viscosity. In some embodiments, stabilization treatment compound (such as PGG) solution is radiopaque and infused to fill the balloon 801.

In some embodiments, the balloon 801 is adapted to make circumferential contact with the thoracic aorta lumen. In some embodiments, once fully apposed, additional infusion would allow for fluid to escape through the peripheral perforations 806 and allow for delivery of stabilization treatment compound into the target tissue. In one example, the first proximal catheter 903 is in fluid communication with the outer balloon 801 covering via a second lumen with fluid communication on the proximal end.

FIG. 8C depicts a cross section view of FIG. 8A along an axis perpendicular to a longitudinal axis of the aorta. In some embodiments, the peripheral walls of the balloon 801 comprise one or more perforations 806. In some embodiments, the balloon 801 is placed within the true lumen near an inlet flap 875 of an aortic tear or dissection. In the embodiment of FIG. 8C, the balloon 801 is inflated to contact the vessel. In the embodiment of FIG. 8C, the inner scaffold 810 creates an inner perfusion lumen 807 for bypass of fluid flow.

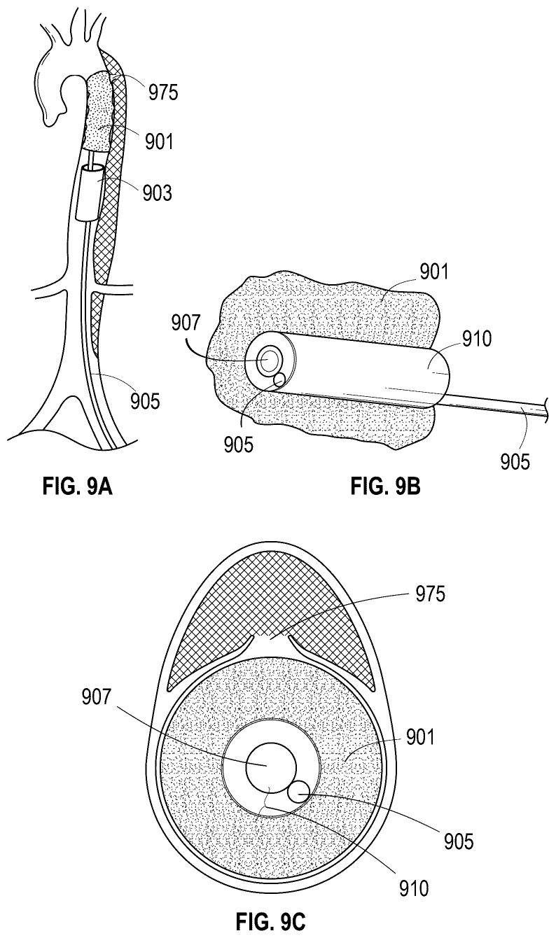

The embodiment depicted in FIGS. 9A-9C can be considered an alternative example of the embodiment depicted in FIGS. 8A-8C. The example of FIGS. 9A-9C is substantially eccentric. In some embodiments, as seen in the oblique partial cutaway view of FIG. 9B, the first proximal end includes a catheter tube 905 designed to inflate the balloon 901 structure. In some embodiments, a proximal catheter tube 903 is larger and is designed to encapsulate the entire structure for deployment and removal. In some embodiments, the catheter tube 905 is integral to the entire length of the balloon 901.

In some embodiments, the balloon 901 has an inner section and outer section with inflation occurring in between. In some embodiments, a middle structure 910 including an inner scaffold and outer scaffold can facilitate a tubular perfusion lumen 907 for fluid flow. In some embodiments, the middle structure 910 includes only an inner scaffold which facilitates a tubular perfusion lumen 907 for fluid flow. In some embodiments, the middle structure 910 is made of a woven material, mesh-like material, a singular spirally shaped wire, or a multiplicity of wires in a longitudinal arrangement. In some embodiments, the middle structure 910 is self-expanding or actuated proximally in order to expand outward. In some embodiments, the middle structure 910 is expanded in a temporary fashion and can be retracted.

In some embodiments, the elastomeric balloon 901 structure is perforated to allow for displacement of blood and delivery of stabilization treatment solution (such as PGG) at or near the target vasculature. In some embodiments, and without being bound by theory, the size of the perforations 906, quantity and viscosity of the stabilization treatment solution influence the rate of delivery. In some embodiments, a perforation 906 size between 0.0015 and 0.005 inches in diameter allows for the preferential inflation of the balloon 901 while experiencing minimal deflation due to blood vessel pressure. In some embodiments, and without being bound by theory, the displaced blood is facilitated with pressure being diverted into the supported lumen 907 structure and upon apposition of the balloon 901 to the wall, additional infusion into the balloon 901 will force liquid to weep from the perforations 906 and allow for vessel wall treatment.

In some embodiments, once delivery is complete, deflation would be through the same fluid communication channel within the proximal catheter tube 905. In the embodiment of FIGS. 9A-9B, the proximal end of the middle structure 910 is attached to the proximal catheter tube 905, such that pulling on the proximal of the end of the proximal catheter tube 905 causes the middle structure 910 to collapse downward and forward as it is pulled proximally for removal of the infusion catheter 900 from the vessel. In some embodiments, when pulled from the distal ends, the middle structure 910 would expand outward.

FIG. 9C depicts a cross section view of FIG. 9A along an axis perpendicular to a longitudinal axis of the aorta. In some embodiments, the peripheral walls of the balloon 901 comprise one or more perforations 906. In some embodiments, the balloon 901 is placed within the true lumen near an inlet flap 975 of an aortic tear or dissection. In the embodiment of FIG. 9C, the balloon 901 is inflated to contact the vessel. In the embodiment of FIG. 9C, the middle structure 910 includes an inner perfusion lumen 907 for bypass of fluid flow. In the embodiment of FIG. 9C, the catheter tube 905 is integral to the entire length of the balloon 901.

### External Delivery:

In some embodiments, the stabilizer treatment compound is delivered externally to the diseased thoracic aneurysm. This can be achieved via a CT guided needle (also ultrasound guided needle), video assisted thoracoscopic surgery (VATS), or topically as part of an open surgical repair. With a CT guided needle, the stabilizer treatment compound is injected through a needle from a posterior approach into the thoracic cavity.

### Stabilizing Compound

Some embodiments described herein include delivery of a stabilizing treatment compound. In some embodiments, the stabilizing treatment compound includes a stabilizing agent. In some embodiments, the stabilizing agent is a phenolic compound. Phenolic compounds are a diverse group of materials that have been recognized for use in a wide variety of applications. For instance, they naturally occur in many plants, and are often a component of the human diet. Phenolic compounds have been examined in depth for efficacy as free radical scavengers and neutralizers, for instance in topical skin applications and in food supplements. Phenolic compounds are also believed to prevent cross-linking of cell membranes found in certain inflammatory conditions and are believed to affect the expressions of specific genes due to their modulation of free radicals and other oxidative species (see, for example, U.S. Pat. No. 6,437,004 to Perricone, incorporated herein by reference in its entirety). In some embodiments, the stabilizing agent includes a polyphenolic compound.

In some embodiments, the stabilizing treatment compound includes one or more excipients. The stabilizing treatment compound excipients may optionally comprise a poloxamer. In some embodiments, the poloxamer allows the fluid stabilizing treatment compound to solidify into a gel within the false lumen due to local temperatures higher than 80° F. The poloxamer may be delivered through a catheter with at least one perforation at the distal end of the catheter, within the false lumen or within the true lumen. In some embodiments, once solidified, the stabilizing treatment compound elutes the stabilizing agent into the diseased aortic tissue. In some embodiments, the poloxamer biodegrades after at least 15 minutes of residence within the false lumen or true lumen. In some embodiments, the solid poloxamer's degradation assists with the dispersion of the stabilizing agent. One example of a suitable material is Pluronic F-127, available from Sigma Aldrich. Another example of a suitable poloxamer is LeGoo® (a tradename of an internal vessel occluder poloxamer composition produced by Pluromed, Inc.).

In some embodiments, the stabilizing agent comprises PGG. Certain risks associated with treatment of thoracic aortic dissections can be mitigated by delivery of pentagalloyl glucose (PGG), such as 1,2,3,4,6-pentagalloyl glucose, to the implantation site, to the repair site, or to the surgical site. For example, PGG can be delivered behind an existing stent graft using a microcatheter or a weeping balloon or can be delivered prior to deployment of a stent graft.

In preferred embodiments, the PGG may be 1,2,3,4,6-pentagalloyl glucose. However, PGG may refer to any chemical structure encompassed by Formula (I):

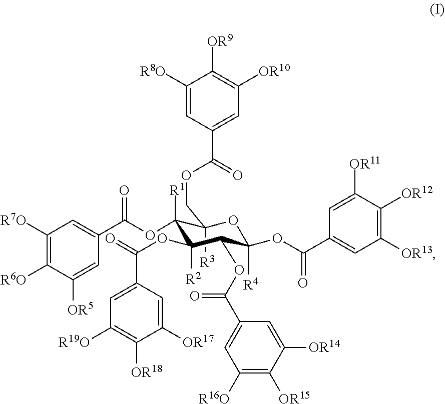

or a pharmaceutically acceptable salt thereof, wherein: R¹-R¹⁹ have any of the values described herein, and wherein the composition is substantially free of gallic acid or methyl gallate. In some embodiments, substantially free is less than about 0.5% gallic acid. In some embodiments, substantially free is less than about 0.5% methyl gallate. In some embodiments, R¹, R², R³ and R⁴ are each independently hydrogen or R^{A}; R⁵, R⁶, R⁷, R⁸, R⁹, R¹⁰, R¹¹, R¹², R¹³, R¹⁴, R¹⁵, R¹⁶, R¹⁷, R¹⁸ and R¹⁹ are each independently hydrogen or R^{B}; each R^{A} is independently selected from the group consisting of —OR^{X}, —N(R^{Y})₂, halo, cyano, —C(═X)R^{Z}, —C(═X)N(R^{Y})₂, —C(═X)OR^{X}, —OC(═X)R^{Z}, —OC(═X)N(R′)₂, —OC(═X)OR^{X}, —NR^{Y}C(═X)R^{Z}, —NR^{Y}C(═X)N(R^{Y})₂, —NR^{Y}C(═X)OR^{X}, unsubstituted C₁₋₁₂alkoxy, substituted C₁₋₁₂alkoxy, unsubstituted C₁₋₈alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted C₃₋₁₂ heteroaralkyl, substituted C₃₋₁₂heteroaralkyl, unsubstituted 3-10 membered heterocyclyl, and substituted 3-10 membered heterocyclyl; each R^{B} is independently selected from the group consisting of —C(═X)R^{Z}, —C(═X)N(R^{V})₂, —C(═X)OR^{X}, unsubstituted C₁₋₈ alkyl, substituted C₁₋₈alkyl, unsubstituted C_{6 or 10}aryl, substituted C_{6 or 10}aryl, unsubstituted C₇₋₁₂aralkyl, substituted C₇₋₁₂aralkyl, unsubstituted 5-10 membered heteroaryl, substituted 5-10 membered heteroaryl, unsubstituted 3-10 membered heterocyclyl and substituted 3-10 membered heterocyclyl, or two adjacent R^{B} groups together with the atoms to which they are attached form an unsubstituted 3-10 heterocyclyl, a substituted 3-10 heterocyclyl, unsubstituted 5-10 membered heteroaryl ring or substituted 5-10 membered heteroaryl ring; each X is independently oxygen (O) or sulfur (S); each R^{X} and R^{Y} is independently selected from the group consisting of hydrogen, unsubstituted C₁₋₈alkyl, substituted C₁₋₈alkyl, unsubstituted C_{6 or 10} aryl, substituted C_{6 or 10} aryl, unsubstituted C₇₋₁₂ aralkyl, substituted C₇₋₁₂ aralkyl, unsubstituted 5-10 membered heteroaryl, substituted 5-10 membered heteroaryl, unsubstituted 3-10 membered heterocyclyl and substituted 3-10 membered heterocyclyl; and each R^{Z} is independently selected from the group consisting of unsubstituted C₁₋₁₂ alkoxy, substituted C₁₋₁₂alkoxy, unsubstituted C₁₋₈alkyl, substituted C₁₋₈alkyl, unsubstituted C_{6 or 10} aryl, substituted C_{6 or 10} aryl, unsubstituted C₇₋₁₂ aralkyl, substituted C₇₋₁₂ aralkyl, unsubstituted 5-10 membered heteroaryl, substituted 5-10 membered heteroaryl, unsubstituted 3-10 membered heterocyclyl and substituted 3-10 membered heterocyclyl.

Without being bound by theory PGG is a small molecule and can penetrate thrombus and other tissue barriers for direct delivery into these diseased portions of the vessel. PGG can protect the remaining elastin within the extracellular matrix with a protective coating as well as strengthen the tissue by binding to the elastin.

The concentrations of PGG which may be safely delivered to a patient may be generally proportional to the purity of the PGG. For example, gallic acid, and methyl gallate, are common cytotoxic impurities which may be removed from a source batch of PGG during the purification process. Eliminating the presence of or reducing the concentration of toxic impurities from the delivered PGG may allow higher concentrations of the PGG to be delivered due to the mitigation of the toxic side effects of impurities commonly found in isolated PGG. For instance, studies have shown that substantially 100% pure PGG may be safely delivered at concentrations up to approximately 0.330% (w/v), 95% pure PGG may be safely delivered at concentrations up to approximately 0.125% (w/v), and 85% pure PGG may be safely delivered at concentrations up to approximately 0.06% (w/v). Delivery of PGG in higher concentrations may enhance the amount of uptake of PGG by the target tissue which may increase the efficacy of the PGG treatment. Delivery of PGG in higher concentrations may increase the rate of uptake of PGG by the tissue allowing the same amount of uptake in shorter delivery times. Reducing or minimizing the delivery time may be advantageous for reducing the overall treatment time, and particularly the duration of time for which the aorta is potentially occluded, as described elsewhere herein. Minimization of the treatment time and particularly the duration of blood occlusion may improve the safety and convenience of the treatment procedure and improve patient outcomes.

Unpurified or partially purified PGG may be obtained from any suitable source and purified according to the methods described herein for use as a therapeutic agent. PGG may be extracted from naturally occurring plants such as pomegranate or Chinese gall nut. Extraction and/or isolation methods may entail solvolysis (for example, methanolysis) of tannin or derivative polyphenols as is known in the art. A PGG hydrate is commercially available from Sigma Aldrich (St. Louis, Missouri) at purities greater than or equal to 96%, as confirmed by HPLC. PGG obtained from these sources may undergo additional purification according to the methods described herein to arrive at substantially pure PGG at the purity levels described elsewhere herein.

In some embodiments, PGG is purified by washing a starting batch of PGG (such as a batch that is less than 99% pure) with a solvent. In preferred embodiments, the solvent may comprise diethyl ether. In other embodiments, the solvent may comprise methanol, toluene, isopropyl ether, dichloromethane, methyl tert-butyl ether, 2-butanone, and/or ethyl acetate. In some embodiments, the washing solution may comprise mixtures of the solvents described herein and/or may be mixed with additional solvents. In some embodiments, the starting batch of PGG may be dissolved into a solution. In some embodiments, the PGG may be dissolved in dimethyl sulfoxide (DMSO). In some embodiments, the PGG may be dissolved in any solvent in which the PGG is soluble, and which is not miscible with the washing solution. The PGG solution may be mixed with the washing solution in a flask and the PGG solution and washing solution may be allowed to separate over time. The washing solution may subsequently be separated from the PGG solution, such as by draining the denser solution from the flask or by decanting the less dense solution. In some embodiments, the mixture of the washing solution and PGG solution may comprise a volume-to-volume ratio of at least about 1:1, 1.5:1, 2:1, 3:1, 4:1, 5:1, or 10:1 washing solution-to-PGG solution. In some embodiments, the washing step may be repeated at least 1, 2, 3, 4, 5, 6, 7, 8, 9, or 10 times. In some embodiments, the washed PGG solution may be evaporated upon purification to precipitate the PGG into a dry (solid) form. In some embodiments, the PGG may remain dissolved, but the volume of the solution may be increased or decreased (for example, by evaporation). In some embodiments, the starting batch of PGG may be in a dry (solid) form. The PGG may be crystalized. In some embodiments, the PGG may be lyophilized. In some embodiments, the PGG may be precipitated from solution. In some embodiments, the starting batch of PGG may be placed on filter paper and the washing solution poured over the filter paper into a waste flask. The filtration may be facilitated by application of a vacuum to the waste flask (vacuum filtration). Residual washing solution may be evaporated from the purified batch of PGG. In some embodiments, the washing step may be repeated at least 1, 2, 3, 4, 5, 6, 7, 8, 9, or 10 times. The purity of the PGG may increase with each wash. The washing procedure may be repeated until a desired level of purity is attained.

In some embodiments, washing the PGG may result in a purity of at least approximately 98.5%, 99%, 99.5%, 99.9%, 99.95%, 99.99%, 99.995%, or 99.999% purity. Purity may be measured as the percent mass (w/w) of PGG in a sample. Purity of the PGG may be measured by any standard means known in the art including chromatography and nuclear magnetic resonance (NMR) spectroscopy. In some embodiments, the purified PGG may comprise no more than approximately 0.001%, 0.005%, 0.01%, 0.05%, 0.1%, 0.2%, 0.3%, 0.4%, 0.5%, 0.6%, 0.7%, 0.8%, 0.9%, or 1% gallic acid. In some embodiments, the purified PGG may comprise no more than approximately 0.001%, 0.005%, 0.01%, 0.05%, 0.1%, 0.2%, 0.3%, 0.4%, 0.5%, 0.6%, 0.7%, 0.8%, 0.9%, or 1% methyl gallate.

PGG may be prepared in a solution for delivery as a therapeutic agent to a patient. The PGG may comprise a purity described elsewhere herein. The PGG may have been purified by the methods disclosed elsewhere herein or may have been purified by other means. In some embodiments, the PGG may be dissolved in a hydrolyzer for subsequent delivery to a patient. The hydrolyzer may comprise any solvent or mixture of solvents in which PGG is readily soluble and which is miscible with water. In some embodiments, the hydrolyzer may be ethanol. In some embodiments, the hydrolyzer may be dimethyl sulfoxide (DMSO). In some embodiments, the hydrolyzer may be contrast media. In some embodiments, the hydrolyzer may be a mixture of ethanol, DMSO, and/or contrast media in any proportions. The hydrolyzer may facilitate the dissolution of PGG into a larger aqueous solution, in which the PGG would not normally be soluble at the same concentration without first being dissolved into the hydrolyzer. The PGG may ultimately be dissolved into a non-toxic aqueous solution suitable for delivery, such as intravascular delivery, to a patient. The aqueous solution may be a saline solution, as is known in the art, or another aqueous solution comprising salts configured to maintain physiological equilibrium with the intravascular environment. The volumetric ratio of the hydrolyzer to the saline solution may be minimized, while maintaining a sufficient volume of hydrolyzer to fully dissolve the desired amount of PGG, to minimize any harmful or toxic effects of the hydrolyzer on the patient, particularly when delivered intravascularly. In some embodiments, the volume-to-volume ratio of saline to hydrolyzer may be no less than about 10:1, 25:1, 50:1, 75:1, 100:1, 200:1, 300:1, 400:1, 500:1, 600:1, 700:1, 800:1, 900:1, or 1000:1. The total volume of the hydrolyzer and saline mixture (including any other additional components) may be configured to prepare the PGG to a desired therapeutic concentration, such as the concentrations described elsewhere herein. In some embodiments, the PGG may be dissolved into the saline or other aqueous solution without a hydrolyzer. In some embodiments, the saline may be warmed (for example, to above room temperature or above physiological temperature) to dissolve or help dissolve the PGG (or other therapeutic agent). For instance, the saline may be warmed to at least about 25° C., 30° C., 35° C., 40° C., 45° C., 50° C., 55° C., or 60° C. prior to dissolving the PGG. In some implementations, the therapeutic solution may be raised to and/or maintained at an elevated temperature (for example, physiological temperature) during delivery.

In some embodiments, PGG (for example, purified PGG) for a therapeutic treatment, including but not limited to those described elsewhere herein, may be provided in a kit comprising the components necessary to prepare the PGG for delivery in a therapeutic solution. In some embodiments, the kit may comprise the PGG in a solid (dry) form, the hydrolyzer, and/or the saline solution. The kit may be configured to optimize the storage conditions of the PGG, for short or long-term storage. In some embodiments, the kit may be configured to store the PGG for up to at least 1 week, 2 weeks, 3 weeks, 4 weeks, 1 month, 2 months, 3 months, 4 months, 5 months, 6 months, 1 year, 2 years, or 3 years. The kit may comprise one or more aliquots of each component in pre-measured amounts or volumes. Each component may be provided in a sealed vial, tube, or other container as is known in the art. The containers may each comprise plastic and/or glass. The containers may be configured (for example, tinted or covered) to protect the components from light and/or other radiation. In some embodiments, the kit may be configured for shipping. For example, the components may be contained in a box or other container including desiccants and/or may be configured for temperature control. In some embodiments, the PGG and/or other components may be supplied in a container that has been purged of air (particularly, oxygen). The component may be stored under vacuum or may be purged with an inert gas, such as nitrogen or argon. In some embodiments, the PGG may be mixed with an antioxidant or other stabilizer, in addition to or alternatively to purging the air. In some embodiments, the antioxidant may comprise Vitamin C, Vitamin E, and/or any other antioxidant or stabilizer which is known in the art and is safe for treatment. In some embodiments, the PGG may be provided already dissolved in the hydrolyzer to a predetermined concentration. In some embodiments, the volume of saline provided may be configured to prepare the PGG at a desired therapeutic concentration. In some embodiments, the volume of saline may be configured to prepare the PGG at a maximal therapeutic concentration, such that a user may dilute the PGG with additional solvent to the desired therapeutic concentration. In some embodiments, the total volume of saline may be configured to prepare the PGG at a concentration below the desired concentration and the user may use only a portion of the volume of the saline to prepare the PGG to the desired concentration. The container of saline may have volume indicators for facilitating measurement of the saline. In some embodiments, the saline may be provided in a plurality of aliquots having the same and/or different volumes, which may allow the user to select an aliquot of a desired volume to prepare the PGG at a desired concentration and/or combine various volumes to prepare the PGG at a desired concentration. In some embodiments, the kit may comprise one or more additional components. For example, the kit may comprise a contrast agent for mixing with the therapeutic PGG solution for allowing indirect visualization of the therapeutic solution, as described elsewhere herein.

In some embodiments, the stabilizing treatment compound includes a contrast media agent. In some embodiments, the operation may be performed under indirect visualization, such as radioscopy. A suitable contrast agent for the method of visualization, (for example, radiocontrast media for radioscopy) may be injected into the blood stream prior to and/or during the operation to visualize blood flow. Accordingly, the occlusion of the blood flow may be visually assessed by indirect visualization.

Considerations for delivery are media types and duration. The lower extremities need to maintain perfusion within minimal interruptions to blood flow. Pressures increase while ascending towards the aortic arch. Different media types may influence the concentration and time for stabilizing compounds such as PGG to deliver a therapeutic dosage into the targeted tissues.

### Terminology and Non-Limiting Details

The term “subject” as used herein, is a broad term, and is to be given its ordinary and customary meaning to a person of ordinary skill in the art (and is not to be limited to a special or customized meaning), and refers without limitation to a human or a non-human mammal, for example, a dog, a cat, a mouse, a rat, a cow, a sheep, a pig, a goat, a non-human primate or a bird, for example, a chicken, as well as any other vertebrate or invertebrate.

The term “mammal” as used herein is a broad term and is to be given its ordinary and customary meaning to a person of ordinary skill in the art (and is not to be limited to a special or customized meaning) and is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

“Treat,” “treatment,” or “treating,” as used herein are broad terms, and are to be given their ordinary and customary meaning to a person of ordinary skill in the art (and are not to be limited to a special or customized meaning), and refer without limitation to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

In some embodiments, and effective amount of stabilizing agent is administered. An “effective amount” or a “therapeutically effective amount” are broad terms, and are to be given their ordinary and customary meaning to a person of ordinary skill in the art (and are not to be limited to a special or customized meaning), and refer without limitation to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

In some implementations, the system comprises various features that are present as single features (as opposed to multiple features). For example, in one embodiment, the system includes a single endoprosthesis, a single infusion catheter, and a single stabilizing treatment compound. In another aspect, the system includes a single flow diverting catheter and a single stabilizing treatment compound. Multiple features or components are provided in alternate embodiments.

In some implementations, the system comprises one or more of the following: means for infusion of a treatment compound (for example, an infusion catheter, a tube with perforations, a weeping balloon, a balloon coupled with a central passage to an endoprosthesis, or another delivery device or delivery system), means for diverting flow through an aortic aneurysm (for example, an endoprosthesis, a stent, a flow diverting catheter), means for stabilizing aortic tissue (for example, a stabilizing treatment compound, a stabilizing agent such as a polyphenolic compound, one or more excipients such as poloxamer), means for occluding blood flow in an aorta (for example, a balloon, a ring covered with a flexible pre-shaped material, a tapered cone with an open cylindrical outlet), etc.

Although certain embodiments and examples have been described herein, aspects of the methods and devices shown and described in the present disclosure may be differently combined and/or modified to form still further embodiments. Optional features of various device and system embodiments may be included in some embodiments and not in others. Additionally, the methods described herein may be practiced using any device suitable for performing the recited steps. Further, the disclosure (including the figures) herein of any particular feature, aspect, method, property, characteristic, quality, attribute, element, or the like in connection with various embodiments can be used in all other embodiments set forth herein. Any section headings used herein are merely provided to enhance readability and are not intended to limit the scope of the embodiments disclosed in a particular section to the features or elements disclosed in that section.

While the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited and in other alternative embodiments one or more method steps may be skipped altogether. The methods disclosed herein include certain actions taken by a practitioner; however, they can also include any third-party instruction of those actions, either expressly or by implication.

Various embodiments of the disclosure have been presented in a range format. It should be understood that the description in range format is merely for convenience and brevity and should not be construed as an inflexible limitation on the scope of the disclosure. The ranges disclosed herein encompass any and all overlap, sub-ranges, and combinations thereof, as well as individual numerical values within that range. For example, description of a range such as from 2 to 21 days should be considered to have specifically disclosed subranges such as from 2 to 5 days, from 2 to 10 days, from 2 to 20 days, from 5 to 15 days, overlapping ranges thereof, as well as individual numbers within that range, for example, 3, 4, 6, 12, 17, 18.5, 19.5 and any whole and partial increments therebetween. Language such as “up to,” “at least,” “greater than,” “less than,” “between,” and the like includes the number recited. Numbers preceded by a term such as “about” or “approximately” include the recited numbers. For example, “approximately 30-50%” includes 30% and 50%. The terms “generally” and “substantially” as used herein represent an amount close to the stated amount that still performs a desired function or achieves a desired result. As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “21” is disclosed, then “about 21” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (for example, where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that when a feature or element is referred to as being “connected,” “attached” or “coupled” to another feature or element, it can be directly connected, attached, or coupled to the other feature or element or intervening features or elements may be present. In contrast, when a feature or element is referred to as being “directly connected,” “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

Terminology used herein is for the purpose of describing particular embodiments only and is not intended to be limiting of the disclosure. For example, as used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

1. A method of treating an aortic tear or dissection of a subject, the method comprising:
inserting an endoprosthesis near an inlet flap of an aortic tear or dissection;
inserting an infusion catheter near the inlet flap of the aortic tear or dissection;
positioning said infusion catheter between an outer surface of the endoprosthesis and an aortic wall;
expanding the endoprosthesis so that the infusion catheter is in contact with aortic tissue near the inlet flap;
infusing a stabilization treatment compound through the infusion catheter, said stabilization treatment compound comprising a contrast media agent, a stabilizing agent, and one or more excipients; and
removing the infusion catheter from the subject.

2. The method of claim 1, wherein inserting the infusion catheter is performed prior to inserting the endoprosthesis.

3. The method of claim 1, wherein inserting the endoprosthesis is performed prior to inserting the infusion catheter.

4. The method of claim 1, wherein contact with the aortic wall comprises contact with the inlet flap.

5. The method of claim 1, wherein contact with the aortic wall does not comprise contact with the inlet flap.

6. The method of claim 1, wherein the stabilizing agent comprises a polyphenolic compound.

7. The method of claim 1, wherein the infusion catheter comprises a perforated balloon.

8. The method of claim 1, wherein the infusion catheter comprises one or more perforated linear catheters.

9. The method of claim 8, wherein the infusion catheter comprises two or more perforated linear catheters.

10. A method of treating an aortic tear or dissection of a subject, the method comprising:
coupling a balloon with a central passage to an endoprosthesis, wherein an outer wall of said balloon comprises perforations or pores;
inserting said endoprosthesis near an inlet flap of an aortic tear or dissection;
expanding the endoprosthesis so that the balloon is in contact with aortic tissue;
infusing a stabilization treatment compound through the balloon so that stabilization treatment compound weeps onto aortic tissue, said stabilization treatment compound comprising a contrast media agent, a stabilizing agent, and one or more excipients; and
deflating and removing the balloon.

11. The method of claim 10, wherein coupling the balloon to the endoprosthesis comprises preloading the balloon into a coaxial restraining sleeve and inserting the endoprosthesis through the balloon, and wherein the method further comprises removing the restraining sleeve, thereby allowing the endoprosthesis to push the balloon into position against an aortic wall.

12-13. (canceled)

14. A method of treating an aortic tear or dissection, wherein the aortic tear or dissection comprises an inlet flap leading to a false lumen of a thoracic aorta separate from a true lumen of the thoracic aorta, the method comprising:
placing a distal end of an infusion catheter inside the false lumen of the thoracic aorta,
placing a stent in the true lumen of the thoracic aorta, and
delivering a stabilization treatment compound through the infusion catheter into the false lumen, said stabilization treatment compound comprising a contrast media agent, a stabilizing agent, and one or more excipients,
wherein said one or more excipients comprises solid poloxamer configured to biodegrade after at least 15 minutes of residence within the false lumen, wherein degradation of the solid poloxamer facilitates dispersion of the stabilizing agent.

15-17. (canceled)

18. The method of claim 14, wherein the infusion catheter comprises a tube with perforations in a distal portion of the infusion catheter.

19. The method of claim 14, wherein a distal portion of the infusion catheter comprises a perforated balloon.

20. A system for treatment of an aortic tear or dissection comprising:
an endoprosthesis;
an infusion catheter shaped to facilitate passage through or behind the endoprosthesis and extending beyond a distal end of the endoprosthesis; and
a stabilizing treatment compound comprising a contrast agent, a stabilizing agent, and one or more excipients.

21-29. (canceled)

30. A method of treating a thoracic aortic aneurysm, comprising:
placing a flow diverting catheter within a lumen of a thoracic aorta near a thoracic aortic aneurysm, said flow diverting catheter comprising an occlusive element on a proximal and/or distal end of the flow diverting catheter;
infusing a stabilizing treatment compound to penetrate an aneurysmal wall, said stabilizing treatment compound comprising a contrast media agent, a stabilizing agent, and one or more excipients;
aspirating remaining stabilizing treatment compound; and
removing the flow diverting catheter.

31-36. (canceled)

37. A system for treatment of a thoracic aortic aneurysm comprising:
a flow diverting catheter comprising an occlusive element on a proximal and/or distal end of the flow diverting catheter; and
a stabilizing treatment compound comprising a contrast media agent, a stabilizing agent, and one or more excipients.

38-41. (canceled)

42. A method of treating an aortic tear or dissection of a subject, the method comprising:
inserting an infusion catheter near an inlet flap of the aortic tear or dissection;
expanding the infusion catheter so that the infusion catheter is in contact with aortic tissue near the inlet flap;
infusing a stabilization treatment compound through the infusion catheter, said stabilization treatment compound comprising a contrast media agent, a stabilizing agent, and one or more excipients; and
removing the infusion catheter from the subject.

43-48. (canceled)

49. A method of treating an aortic tear or dissection of a subject, the method comprising:
inserting an infusion catheter comprising two or more elongated balloons near an inlet flap of the aortic tear or dissection;
expanding the infusion catheter so that the infusion catheter is in contact with aortic tissue near the inlet flap;
wherein the two or more elongated balloons are coated with a stabilization treatment compound comprising a stabilizing agent and one or more excipients; and
removing the infusion catheter from the subject.

50-52. (canceled)

53. A method of treating an aortic tear or dissection of a subject, the method comprising:
inserting an infusion catheter comprising a catheter that may be actuated to form a spiral near an inlet flap of the aortic tear or dissection;
expanding the infusion catheter by actuating the catheter to form a spiral such that the infusion catheter is in contact with aortic tissue near the inlet flap;
infusing a stabilization treatment compound through the infusion catheter, said stabilization treatment compound comprising a contrast media agent, a stabilizing agent, and one or more excipients;
inserting an endoprosthesis within loops of the spiral,
removing the infusion catheter from the subject; and
deploying the endoprosthesis near the inlet flap of an aortic tear or dissection.

54. (canceled)
